(19) United States
(12) Patent Application Publication
(10) Pub. No.: US 2025/0382081 A1
(43) Pub. Date: Dec. 18, 2025

(54) POLYMER FILM ENVELOPE PACKAGE FOR COMPACT FEMININE HYGIENE PAD PRODUCT, AND PROCESS AND TOOLING FOR MANUFACTURE THEREOF

(71) Applicant: The Procter & Gamble Company, Cincinnati, OH (US)

(72) Inventors: Chad BURNETT, Cincinnati, OH (US); Dale Francis BITTNER, Harrison, OH (US); Mathias Johannes HILPERT, Mason, OH (US); Clarissa MALDONADO, Cincinnati, OH (US); Curtis Hunter VANVALKENBURGH, Batavia, OH (US)

(21) Appl. No.: 18/743,219

(22) Filed: Jun. 14, 2024

(51) Int. Cl. B65B 9/087; A61F 13/551; B65B 51/16; B65B 51/26; B65D 27/00

(52) U.S. Cl. CPC B65B 9/067; B65B 51/16; B65B 51/306; A61F 13/5514; B65B 51/26; B65D 27/00

(57) ABSTRACT

A compactly packaged product, and equipment and process for forming the package, are disclosed. The product is contained in an envelope package formed of polymeric film, with a pair of substantially straight parallel oppositely-disposed side seams defining an enveloped width therebetween. The process and equipment used provide for the enveloped width to be relatively close to the width of the contained product, and for effectively secure seams of relatively narrow width. The equipment and process includes a pair of rollers with respective seaming and severing elements oriented along a cross direction, and forming a nip therebetween. The equipment is configured to simultaneously form secure seams in, and effectively sever, flow wrapping about respective leading and trailing products, and thereby effectively sever completed leading packages from trailing packages as the flow-wrapped products move through the nip. The equipment and process enables the described functions at a relatively high throughput rate.

### BACKGROUND

Users/consumers of feminine hygiene pads have developed a number of varying expectations and preferences for such products over the years, as the products themselves have evolved. These expectations and preferences include (in no particular order) (1) that the pad have suitable absorption performance such that it will readily accept, absorb, contain, isolate and effectively retain all menstrual fluid discharged, away from the user's skin and without leaking, over a reasonable time of use/wear; (2) that the pad be as thin (low-caliper/non-bulky), flexible and pliable as possible for purposes of comfort, accommodation of the wearer's body movements, and discreetness of wear under clothing; and (3) that the pad and its packaging provide for convenient and discreet carry, and discreet and easy package opening and access to the pad at the time it is needed (particularly when the user is away from home). Many women may have anxiety about leaving the home during menstruation, and about being sufficiently prepared, while away from home, to effectively address needs relating to absorption and containment of discharged menstrual fluid. In addition to desiring a sufficiently absorbent pad that can be worn comfortably and discreetly under clothing, many women desire discreetness and convenience with respect to having to carry pads with them outside the home, and with respect to having to open and access a packaged pad for use, in situations in which they need to use a bathroom or restroom in an unfamiliar, non-private or otherwise uncomfortable place and/or circumstances.

For the purposes of responding to these market demands, individually, compactly-packaged pads have been developed. These products, however, present opportunities for product design, process design and processing equipment design, to enable assembly of individual pad packages that are as compact as possible; to maximize efficiency of packaging material usage; to minimize incidence of package failure (e.g., package seam failure); and to enable packaging at commercially feasible and desirable, efficient production rates. There is conflict among these objectives, and meeting all of them simultaneously presents challenges.

### BRIEF DESCRIPTION OF THE DRAWINGS

FIG. 1 is a plan view of a feminine hygiene pad, open and laid out flat, topsheet side facing the viewer.

FIG. 2A is a schematic lateral cross section of the feminine hygiene pad of FIG. 1, taken along line 2A-2A in FIG. 1.

FIG. 2B is a view of portion 2B of the drawing of FIG. 2A, enlarged to depict sublayers of an absorbent layer.

FIG. 3 is a plan view of the feminine hygiene pad of FIG. 1, shown with side portions folded laterally over and inwardly alongside longitudinal fold lines.

FIG. 4A is a schematic side view of a feminine hygiene pad folded in four sections in a roll fold configuration, with a main fold nose oriented toward the top of the figure.

FIG. 4B is a schematic side view of a feminine hygiene pad folded in four section in a book jacket fold configuration, with a main fold nose oriented toward the top of the figure.

FIG. 4C is a schematic front view of a folded feminine hygiene pad, with a main fold nose oriented toward the top of the figure.

FIG. 5A is a front view of an envelope package in an opened configuration.

FIG. 5B is a schematic cross section of the package as shown in FIG. 5A, taken along line 5B-5B in FIG. 5A.

FIG. 6A is a front view of an envelope package in a closed configuration.

FIG. 6B is a schematic cross section of the package as shown in FIG. 6A, taken along line 6B-6B in FIG. 6A.

FIG. 7 is a front view of the closed envelope package as shown in FIG. 6A, showing positioning of a folded feminine hygiene pad within the package.

FIG. 8 is a front view of the envelope package as shown in FIG. 5A, shown in an opened configuration and showing positioning of a folded feminine hygiene pad within the package.

FIG. 9A is a front view of another example of an envelope package in an opened configuration.

FIG. 9B is a schematic cross section of the package as shown in FIG. 9A, taken along line 9B-9B in FIG. 9A.

FIG. 10A is a front view of an another example of an envelope package in a closed configuration.

FIG. 10B is a schematic cross section of the package as shown in FIG. 10A, taken along line 10B-10B in FIG. 10A.

FIG. 10C is a front view of an another example of an envelope package in an opened configuration.

FIG. 10D is a front view of an another example of an envelope package in a closed configuration.

FIG. 11A is a schematic plan view depiction of an example of process flow for finishing and packaging feminine hygiene pads.

FIG. 11B is a schematic plan view depiction of an alternative example of a portion of the process flow depicted in FIG. 11A.

FIG. 12 is a schematic side view depiction of operating components of an example of a separation unit, shown operating with a series of folded pads.

FIG. 13A is an expanded plan view depiction of a portion of backsheet adhesive coversheet supply web with separation perforations.

FIG. 13B is an expanded plan view depiction of a portion of the backsheet adhesive coversheet supply web shown in FIG. 13A, following separation along separation perforations to form a backsheet adhesive coversheet.

FIG. 14A is a plan view of a portion of continuous package film stock bearing applied flap stickers.

FIG. 14B is a cross section of the package film stock depicted in FIG. 14A, taken along line 14B-14B shown in FIG. 14A.

FIG. 15 is a view of the cross section of FIG. 14B, further depicting a location of disposition of a folded pad relative thereto, in preparation for flow wrapping.

FIG. 16 is an expanded cross section view of the elements depicted in FIG. 15, following their exit from a flow-wrap unit.

FIG. 17 is a schematic cross section and side view depicting a portion of a stream of feminine hygiene pads wrapped in package film, passing through a nip between seaming and severing rollers.

FIG. 18 is a schematic cross section of portions of a pair of seaming and severing rollers.

FIG. 19A is a schematic cross section of a seaming knife and an opposing anvil, respectively forming components of a pair of seaming and severing rollers, shown apart from their respective rollers.

FIG. 19B is a perspective view of the seaming knife and an opposing anvil depicted in FIG. 19, shown apart from their respective rollers.

FIG. 20 is an expanded schematic cross section of portions of the seaming knife and opposing anvil shown in FIG. 19.

FIG. 21 is the expanded schematic cross section of the portions of the seaming knife and opposing anvil shown in FIG. 20, further shown acting upon respective portions of polymeric film as it passes between the rollers.

FIG. 22 is an expanded schematic cross section view of respective portions of polymeric film that have been affixed together and severed away to form an edge seam of the portions.

FIG. 23 is a black-and-white/grayscale reproduction of a photograph taken via scanning electron microscopy, depicting a magnified image of a perspective view including a cut cross section of respective layers of polymeric film that have been affixed together and severed away to form an edge seam of the layers.

FIG. 24 is a schematic enlarged schematic plan view of respective portions of polymeric film that have been affixed together and severed away to form an edge seam of the portions.

FIG. 25 is a schematic facing view of an envelope package containing a folded feminine hygiene pad.

FIG. 26 is a schematic lateral cross section showing a folded feminine hygiene pad within an envelope package.

FIG. 27 is a schematic enlarged cross section of a portion of a multi-layer film.

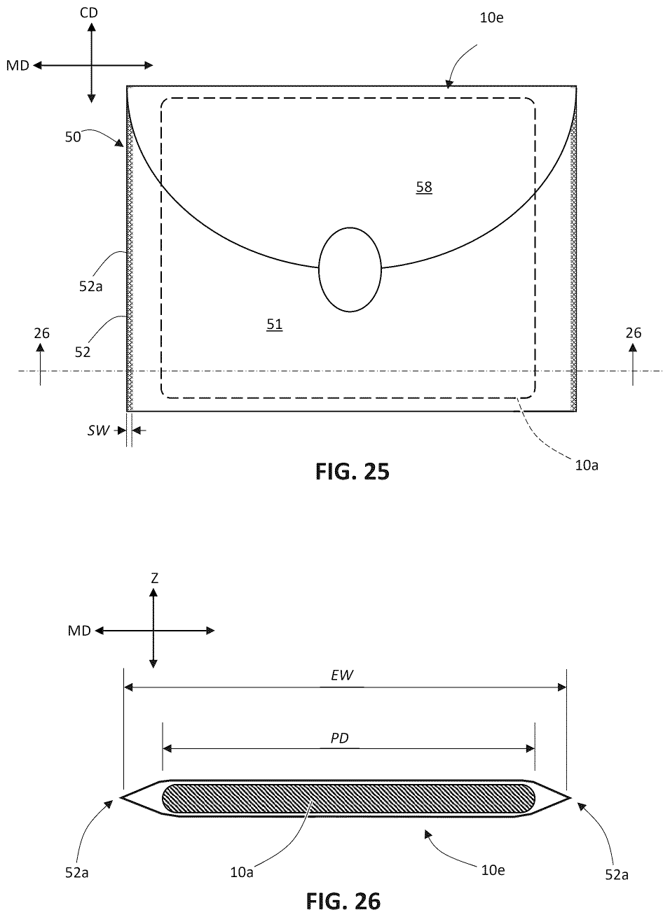

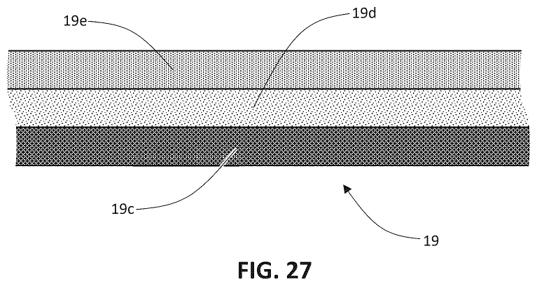

NOTE: With the exception of the reproduction of a photograph in FIG. 25, all figures herein are schematic and are not represented to be proportionately-drawn or scale drawings.

### DESCRIPTION OF EXAMPLES

### Definitions

With respect to a feminine hygiene pad that is open and laid out flat on a horizontal planar surface, “lateral” refers to a direction perpendicular to the longitudinal direction and parallel the horizontal planar surface. “Width” refers to a dimension measured along a lateral direction.

With respect to a feminine hygiene pad that is open and laid out flat on a horizontal planar surface and having a length measured from its forwardmost end to its rearwardmost end, “longitudinal” refers to a direction parallel with the line along which the length is measured, and parallel to the horizontal planar surface. “Length” refers to a dimension measured in the longitudinal direction.

With respect to a feminine hygiene pad, the terms “front,” “rear,” “forward” and “rearward” relate to features or regions of the pad in a position as it would ordinarily be worn by a user, and the front and rear of the user's body when standing.

With respect to a feminine hygiene pad that is open and laid out flat on a horizontal planar surface, “z-direction” refers to a direction perpendicular to the horizontal planar surface. When the pad is being worn by a user (and thus in a curved configuration), “z-direction” at any particular point location on the pad refers to a direction normal to the wearer-facing surface of the pad at the particular point location.

With respect to a feminine hygiene pad, “wearer-facing” is a relative locational term referring to a feature of a component or structure of the pad that when in use that lies closer to the wearer than another feature of the component or structure that lies along the same z-direction. For example, a topsheet has a wearer-facing surface that lies closer to the wearer than the opposite, outward-facing surface of the topsheet.

With respect to a feminine hygiene pad, “outward-facing” is a relative locational term referring to a feature of a component or structure of the pad that when in use that lies farther from the wearer than another feature of the component or structure that lies along the same z-direction. For example, a topsheet has an outward-facing surface that lies farther from the wearer than the opposite, wearer-facing surface of the topsheet.

The “machine direction” (MD) is the direction along which a flat or planar web material undergoing processing moves through processing equipment. The “cross direction” (CD) is the direction orthogonal to the MD, along a plane (web plane) along which a major surface of the flat or planar web material as it enters the processing equipment. The “Z-direction” is the direction orthogonal to the MD, and orthogonal to the web plane.

A seam in which layers of material are “permanently” affixed to each other is one in which the layers cannot be separated without destruction of the seam and/or damage to the layers.

Description

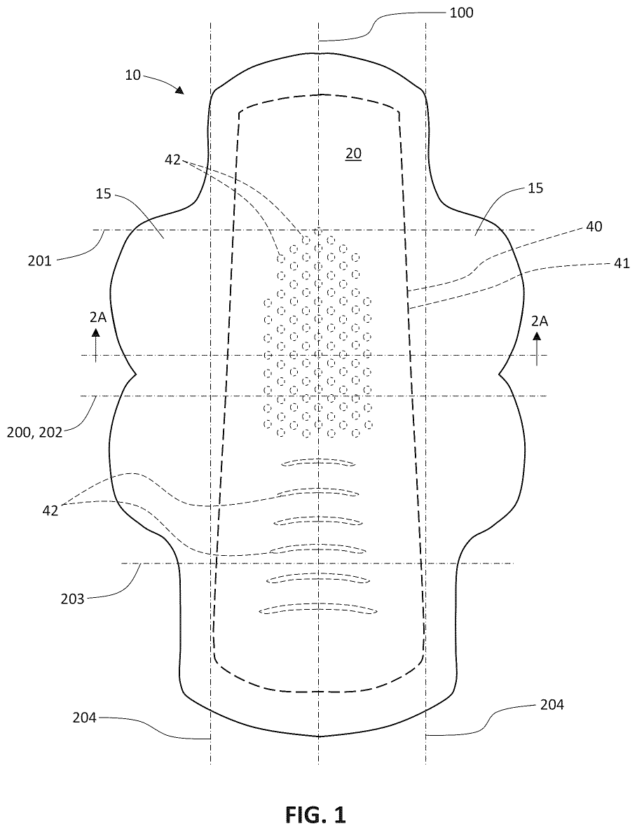

Referring to FIGS. 1, 2A and 3, a feminine hygiene pad 10 may include a liquid permeable topsheet 20, a liquid impermeable backsheet 30 and an absorbent layer 40 disposed between the topsheet and the backsheet. The absorbent layer has an outer perimeter 41. In peripheral regions outside the outer perimeter 41, the topsheet and the backsheet may be bonded together in laminated fashion by any suitable mechanism including but not limited to adhesive bonding, thermal bonding, pressure bonding, etc., thereby retaining and holding the absorbent layer 40 in place between the topsheet 20 and the backsheet 30. Pad 10 may include opposing wing portions 15 extending laterally outside of perimeter 41 by a comparatively greater width dimension than the main portion of the pad. Wing portions 15 may be formed of lateral extensions of the material forming the topsheet 20, backsheet 30, or both together. The outward-facing surface of the backsheet forming the undersides of the main portion and the wing portions may have deposits of adhesive 35 thereon. Adhesive deposits 35 may be provided to enable the user to adhere the pad to the inside of her underpants in the crotch region thereof, and wrap the wing portions through and around the inside edges of the leg openings of the underpants and adhere them to the outside/underside of the underpants in the crotch region, providing supplemental holding support and helping guard the leg edges of the underpants against soiling. When pad 10 is packaged, adhesive deposits 35 may be covered by one or more removable sheets of release film or paper (not shown) that cover the adhesive deposits 35 and shield them from contact with other surfaces until the user is ready to remove the sheets and place the pad for use.

To ensure that the pad facilitates sufficiently compact folding and packaging as described herein, it may be desired to limit the length LP of the pad to no greater than 32 cm, more preferably no greater than 29 cm, and even more preferably no greater than 26 cm. Alternatively, or in addition, it may be desired to limit the length LAL of the absorbent layer (also measured in the longitudinal direction) to no greater than 30 cm, more preferably no greater than 27 cm, and even more preferably no greater than 24 cm.

The pad and it components (including but not limited to topsheet 20, backsheet 30 and absorbent core 40) may have any structure and composition described in, for example, United States applications having Ser. No. 16/750,074; Ser. No. 16/750082; Ser. No. 16/750089; Ser. No. 16/750095; Ser. No. 18/329848 and 62/795745, which are incorporated herein by reference, or any other suitable composition and structure known in the art. These applications describe examples of pads having various features including absorbent cores formed of (High Internal Phase Emulsion (HIPE) foam.

### Folding

Referring to FIGS. 1, 3 and 4A-4C, a particular folding arrangement for the pad, to be placed inside an individual package, may be employed. This folding arrangement can be facilitated in some circumstances by construction of a relatively thin pad utilizing an open-cell foam HIPE material for the absorbent layer, as described in the cited applications.

As depicted in the figures, pad 10 may first be folded about side longitudinal fold lines 204, about which wings 15 are folded laterally over the wearer-facing surface of the pad. Following such folding, one or more suitably-sized section(s) of a thin release film or paper (not shown) may be laid over and applied to the portions of the wings having backsheet adhesive 35 deposited thereon, to cover the adhesive and protect it from unwanted sticking, until the time the user wishes to apply the pad to her underpants. To avoid unnecessarily adding caliper to the pad in the completely folded configuration, it may be desired to avoid folding the pad along any longitudinal fold lines that traverse the absorbent layer or polymeric foam material included in the absorbent layer. As noted above, the central outward-facing surfaces of the pad having backsheet adhesive 35 deposited thereon may also be covered with one or more sections of release film or paper (not shown).

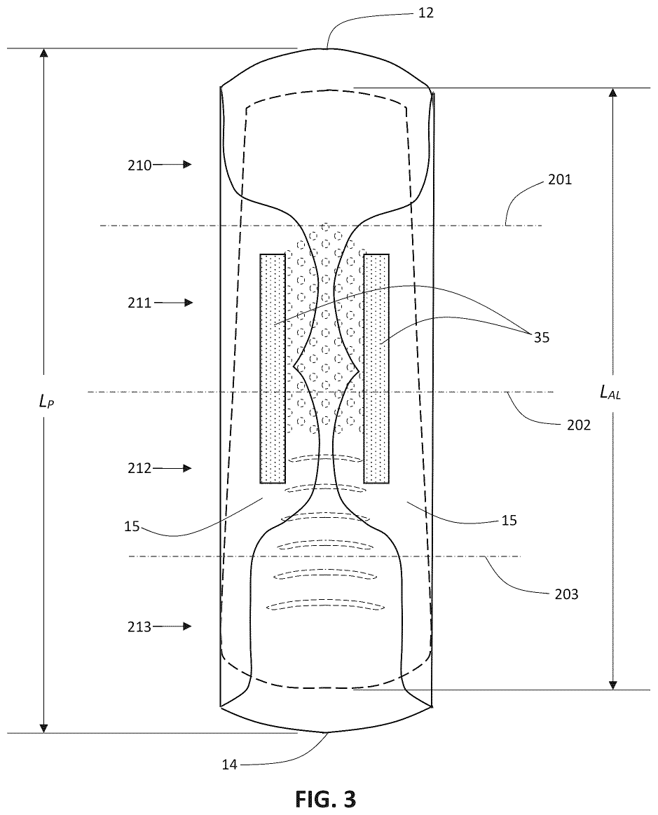

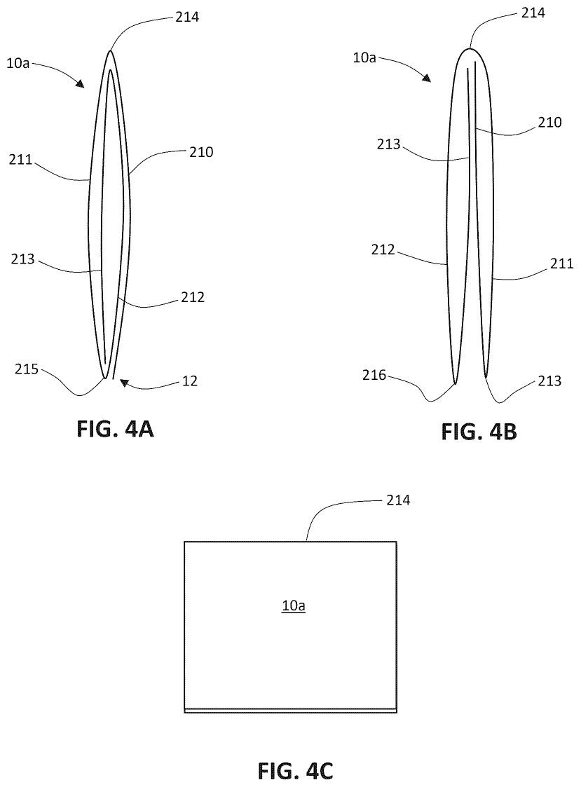

After folding of the wings 15 about side longitudinal fold lines 204, the pad may be further folded into at least two, preferably at least three, and more preferably four sections. If folded into four sections as reflected in FIGS. 3 and 4A-4C, the sections may include front section 210, front-mid section 211, rear-mid section 212 and rear section 213, along at least a front lateral fold line 201, middle lateral fold line 202 and rear lateral fold line 203. Middle lateral fold line 202 may, but does not necessarily have to, coincide with pad lateral axis 200. Sections 210, 211, 212 and 213 may be approximately equal in length, or the lengths of each (i.e., the positions of the lateral folding lines) may be adjusted to facilitate folding of one section length inside another section length.

Along lateral folding lines 201, 202 and 203, the pad may be folded into at least four sections 210, 211, 212 and 213 in a number of ways. However, for dual, but unrelated, purposes of (1) providing a folded pad configuration 10a with a singularized fold nose 214, and (2) minimizing folding damage to an absorbent layer formed of two layers of open-celled HIPE foam material as described in the applications cited above, one of two folding configurations may be applied.

The first is a “roll” fold configuration depicted in FIG. 4A. In a roll fold configuration, rear section 213 is folded along about rear lateral fold line 203 over rear-mid section 212; then both sections 213 and 212 together are folded about middle lateral fold line 202 over front-mid section 211; and lastly, the three section 213, 212 and 211 together are folded about front lateral fold line 201 over front section 210. The foregoing procedure results in a front-section-out roll fold configuration, in which front section 210 and forward end 12 are disposed on the outside of the folded pad configuration 10a as shown in

FIG. 4A. It will be appreciated that an alternative roll fold configuration as described above may be formed by first folding the front section 210 over about front lateral fold line 201, and preceding to fold rearward, resulting in a rear-section-out roll fold configuration, in which rear section 210 and rearward end 14 lie on the outside of the folded configuration. A front-section-out roll fold configuration as shown in FIG. 4A may be desired, however, for purposes of user convenience, because it provides for front-to-rear unfolding that may be more intuitive for the user. It will be appreciated that a front-section-out roll fold configuration, or a rear-section-out roll fold configuration (if desired), may be created via steps that differ from those described above.

The second configuration is a “book jacket” fold configuration, named herein because it resembles the manner in which a book jacket (or book dust jacket) is folded, and is depicted in FIG. 4B. In a book jacket fold configuration, front section 210 is folded about front lateral fold line 201 over front-mid section 211; and rear second 212 is folded about rear lateral fold line 203 over rear-mid section 212 (these two folds can occur simultaneously or one after another in either order). Finally, the two remaining portions comprising front section 210 folded over front-mid section 211 as one portion, and rear section 213 folded over rear-mid section 212, are folded about middle lateral fold line 202 to bring them together to create the book jacket fold configuration depicted in FIG. 4B.

It will be appreciated that in both the roll and book jacket fold configurations depicted and described, in all folds about lateral fold lines 201, 202 and 203, the fold is formed with the wearer-facing (i.e., topsheet) surfaces facing inward in the fold. This serves three purposes. First, it is believed that it provides for more intuitive, and therefore more convenient, unfolding for the user. Second, it cooperates with an absorbent layer formed of two sublayers of open-celled HIPE foam, as described in the applications cited above, to help minimize permanent deformation, or damage, to the absorbent layer resulting from folding.

Third, it can be seen in FIGS. 4A and 4B that both the roll fold configuration and the book jacket fold configuration result in an isolated and singularized main fold nose 214, wherein an outside folded edge along one of the lateral fold lines is alone, with no proximate folded or end edges present-in contrast to the secondary fold nose 215 proximate to end edge 12 shown at the bottom of FIG. 4A and proximate secondary fold noses 216, 217 shown at the bottom of FIG. 4B). A singularized main fold nose 214 may be desired for purposes of user convenience and user perceptions of greater imperviousness to contamination of the product, as will be described below.

### Package

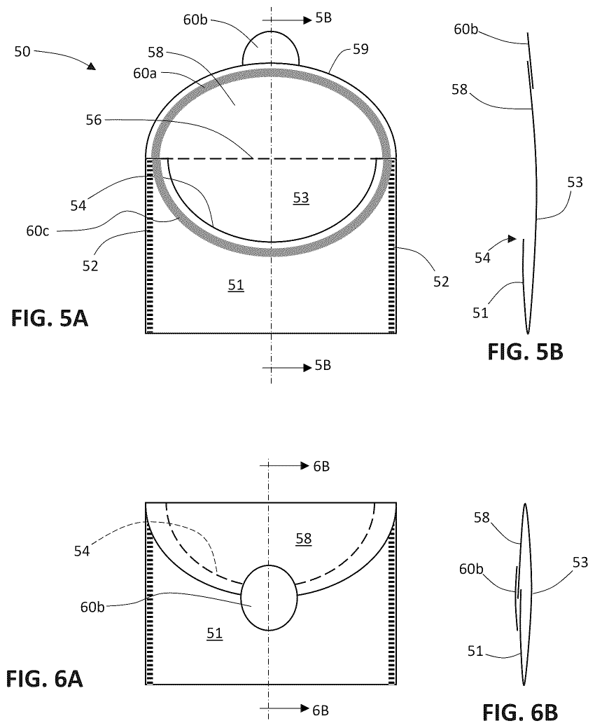

Referring to FIGS. 5A, 5B, 6A, 6B, 7 and 8, for purposes of providing a package for an individual folded feminine hygiene pad that can be easily and quietly opened by a user, it may be desired to provide a package having some or all of the features depicted and described herein. Package 50 may have an envelope configuration as depicted. It may be formed from a single sheet of web material, preferably a nonwoven web material, polymeric film material, or laminate of a nonwoven web material and polymeric film material. The single sheet of material may be cut to appropriate length, size and shape configured to provide front panel 51, rear panel 53 and closure flap 58, wherein front panel 51 is cut to provide an extended access front opening edge 54 visible in an opened configuration shown in FIGS. 5A and 5B, and a closure flap 58 that may in some examples be configured to overlie and cover front opening edge 54 when in the closed configuration as shown in FIGS. 6A and 6B (flap 58 folded about closure flap fold line 56). The cut package material may be folded as shown and then ultrasonically, thermally, compressively and/or adhesively bonded alongside edges thereof to form side seams 52 and thereby form the envelope structure. In order to minimize the size of the package, it may be desired to minimize the margin of film material along the side seams that is present in the seams, and utilize a joining mechanism (e.g., by application of pressure and, if desired, heat, directly or via ultrasonic means) that results in side seams formed that join the front and rear panels of the film material, and that have an average width of from 200 μm to 1,000 μm, or from 200 μm to 800 μm, or from 300 μm to 600 μm, or within these ranges, equal to or less than 500 μm.

Where the package includes a polymeric film material, it may be desired that the film material have properties that make it relatively quiet when handled or manipulated, for purposes of providing a discreet package. In some examples, the film material may comprise polyethylene (PE), and in some examples a weight majority of PE, or 60 percent by weight or more PE. In some examples the polymer resin from which the film is formed may include a particulate filler, a particulate colorant pigment and/or a particulate opacifier, such as titanium dioxide (TiO₂). Without intending to be bound by theory, it is believed that such particulate fillers, in addition to having a whitening or coloring effect, may beneficially reduce noise generation/transmission characteristics of the polymer film. Nonlimiting examples of suitable polymer film and particle filler compositions are disclosed in US 2015/0376384 and US 2015/0376383.

Many currently marketed feminine hygiene pads are individually packaged in packages that require the consumer to destructively tear the package along seams or through the package material itself, to access the pad within. Such destructive opening creates the potential to generate, or actually generates, unwanted noise during package opening. In order to further minimize noise created by opening of the package, and to preserve the package structure so it can be used to receive and hold a used pad for later disposal, it may be desired that the package be configured so that it can be opened without substantial destruction thereto. In some examples, the packaging may be configured such that accessing the pad within requires separating the package material along a total/combined distance of no more than 50 percent of the total length of all seams present, preferably no more than 30 percent of the total length of all seams present, and more preferably no more than 15 percent of the total length of all seams present.

In some examples, an openable closure element may be provided, in combination with any of the other package features described herein. In some examples a closure element may include a releasable flap closure adhesive 60a disposed on the underside of the flap 58, or panel closure adhesive 60c disposed on the outside of the package front panel 51. In some examples the openable closure element may include an adhesive closure tape or sticker 60b that overlies the edge of closure flap 58 and adheres to the front panel 51 of package 50. In some examples the closure element may be a deposit of releasable closure adhesive 60a (e.g., in a position as depicted in FIG. 5A) alone, applied to the underside of flap 58 in a suitable position and shape to adhere the flap to front panel 51 without contacting a pad contained in the package. Such adhesive may be applied in a position on the flap so as to be present along most or substantially the entire opening edge 54 when the flap 58 is closed (i.e., extending substantially along the flap edge and/or opening edge 54 from one side seam 52 to the other), to make a more complete closure that reduces chances of contamination of a new pad within the package. In some examples a deposit of releasable panel closure adhesive 60c may be disposed on the front panel 51, which may be desired to minimize likelihood of inadvertent contact with the pad. In some examples the releasable adhesive may also be reusable/refastenable, which may make the package 50 more useful for storing a used pad for disposal.

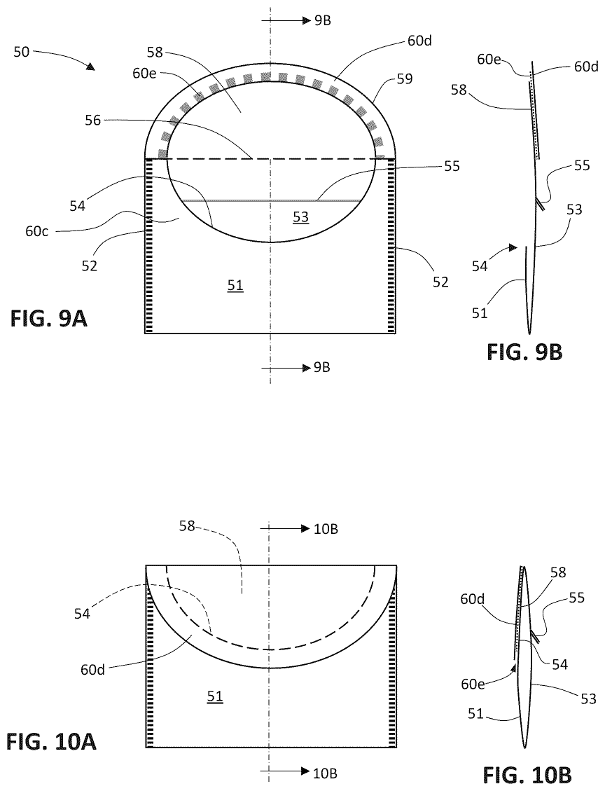

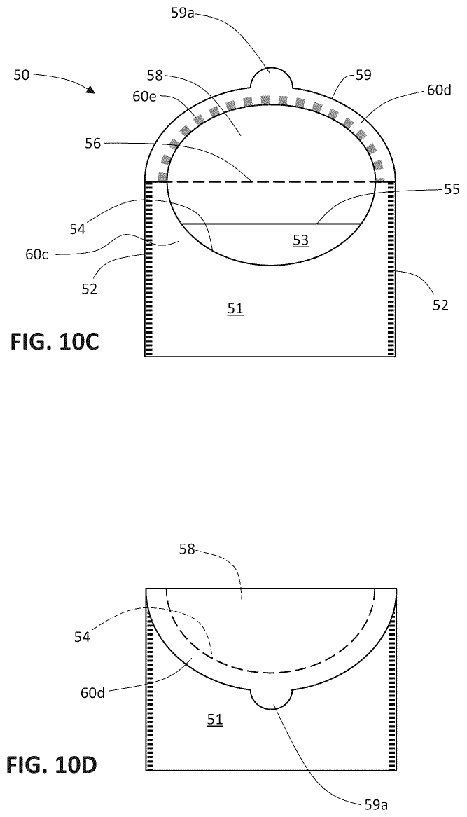

In some examples such as depicted in FIGS. 9A, 9B, 10A and 10B, an envelope package may be formed about a pad in a flow-wrap process in which a seam 55 is created in back panel 53. As depicted in FIGS. 9B and 10B, seam 55 is a fin seam; however, seam 55 may also be in the form of an overlap seam that lies flush along the rear panel 53. Either a fin seam or an overlap seam may be formed and held together via thermal bonding/fusion of the separate portions of film joined therealong, by a deposit of a suitable adhesive between the separate portions of film, or a combination thereof. In some examples it may be desired that attachment between the joined sections forming back panel 53, at seam 55, whether effected by adhesive or by welding/fusion, be intermittent or discontinuous and may have spaced intervals of attachment, rather than being continuous, along seam 55. This may be desired to allow venting of air from the finished package upon compression of the package along a Z-direction, such as may occur in processes downstream of package formation, reducing chances that the package will burst open along seams or along the flap closure.

Still referring to FIGS. 9A, 9B, 10A and 10B, rather than be separate of film forming front panel 51, flap 58 may be contiguous and integral with film forming front panel 51, and may be defined along edge 54 by a path of perforations, laser scoring, mechanical scoring or cutting or other suitable mechanism for imparting a path of separation along which front panel 51 and flap 58 will easily tear away or separate from each other. A larger flap sticker 60d with flap sticker adhesive 60e disposed to hold it to the front panel 51 and flap 58 may overlie the path of separation and thereby prevent opening of the package until desired by the user. When the user wishes to open the package, she may lift and peal the flap sticker 60d upwardly, and the adhered flap sticker 60d will pull the flap 58 along therewith, causing the front panel 51 and flap 58 to separate along the path of separation to create access opening edge 54. In some examples the film forming front panel 51 may be cut (such as by die-cutting) substantially entirely through its thickness along the path of separation during the manufacturing/packaging process, following application of flap sticker 60d to the package film, such that the flap sticker 60d and flap sticker adhesive 60e thereunder are the only structure that effectively holds the flap in a closed position after packaging is completed. This may provide for a particularly quiet flap opening experience for the user, when a suitable flap sticker adhesive composition is included, because the user will not need to tear the film of the front panel (which has a potential to generate noise) to open the package. In some examples it may be desired that the flap sticker be of a size and shape suitable to cover the entirety of the path of separation. Such a feature may serve to enhance the ability of the package to protect a new pad from contamination prior to use; to create favorable consumer perceptions resulting from such effect; and to make the package a more effective container/disposal aid for receiving, isolating and carrying a used pad until the time the user can dispose of it.

As may be appreciated from FIGS. 10C and 10D, in some examples a flap sticker 60d may include a lift tab 59. Lift tab 59 may simply be an extension of the distal end of flap sticker 60d. Lift tab 59 or a distal portion thereof may be provided with a reduced amount, or entirely without, any adhesive 60e thereon. In such configuration, lift tab 59 may easily be lifted away from front panel 51 and grasped by the consumer, facilitating the consumer's lifting and peeling upward of flap sticker 60d and/or flap 58 to open the package.

It will be appreciated that a package configuration such as any of those depicted in FIGS. 5A, 5B, 6A, 6B, 7, 8, 9A, 9B, 10A-10D and described herein, may be configured so as to be openable with substantially limited or even no destruction thereto, and to be recloseable, so as to make it suitable to be reused as a disposal aid-into which the user may insert a used, folded pad, and reclose the package around it, to isolate the used pad until a convenient time for disposal thereof. Where an openable flap 58 is provided, it may be configured to be openable without substantial destruction of the flap itself, and without substantial destruction or separations of other portions of the package (e.g. side seams 52), such that the flap is effectively recloseable to cover the access opening. Additionally, a reclosure refastenability feature may be provided, such as, for example a releasable/refastenable flap closure adhesive 60a beneath the flap or a releasable/refastenable adhesive 60e beneath a flap sticker 60b or 60d, such that the flap 58 and access opening may be fastenably reclosed after opening.

As may be appreciated from the figures, in some examples it may be desired that the opening edge 54 follow a curving path, which will avoid localization of stresses therealong and thereby avoid unintended tearing of the package film. In some examples it may be desired that the curving path be configured such that a greatest portion of the contained folded pad is exposed and made accessible to the user at a location between the side seams 52, for purposes of user convenience.

It may be desired that the package 50 be sized relative the folded pad 10a, so as to be as small as possible (for convenient and discreet carry) without being so snug as to create friction between the folded pad 10a and the insides of the package sufficient to frustrate easy and quiet withdrawal of the pad from the package. Similarly, and also for purposes of nondestructive and easy withdrawal of the pad from the package, it may be desired that the folded pad 10a and the package 50 be configured such that there are few or no substantial deposits of adhesive, preferably no deposits of adhesive, disposed between the outside surfaces of the folded pad 10a and the inside surfaces of the package 50 and/or the closure flap 58.

Through prototyping, it has been discovered that a normal-capacity daytime use feminine hygiene pad of the same design and materials as current ALWAYS INFINITY pads (a product of The

Procter & Gamble Company, Cincinnati, Ohio) and having an absorbent layer formed of an open-celled HIPE foam, can be folded and packaged as described herein to have an uncompressed package caliper when laid flat on a horizontal surface of 25 mm or less, more preferably 20 mm or less, even more preferably 15 mm or less, or from 10 mm to 25 mm, more preferably from 10 mm to 20 mm, and even more preferably from 10 mm to 15 mm—following removal from a larger package containing a plurality of individually packaged pads, and a 24-hour rest period. This package caliper contributes to providing a relatively small, packaged pad believed to be preferred by many consumers for its discreet pocketability and ease of discreet one-hand carry.

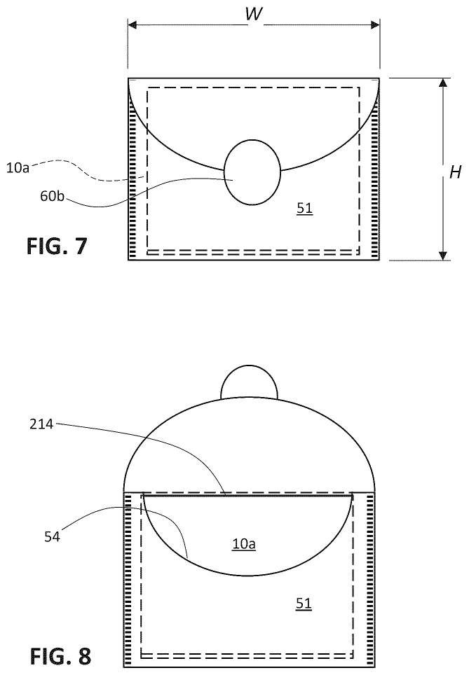

It has been discovered that, between packages that are square or nearly square in shape (aspect ratio of package height to width of approximately 1.0) and packages that are rectangular but not square in shape, consumers prefer the non-square packages, for reasons that are not thoroughly understood but are believed to relate to perceived convenience of pocketability and/or discreet carry. Accordingly, it may be desired to configure the pad and the package such that the closed package with the contained pad is rectangular in shape and has an aspect ratio of height H to width W (see FIGS. 7) of 0.40 to 0.95, more preferably 0.45 to 0.85, and even more preferably 0.50 to 0.75.

In order to maximize user convenience, it may be desired that a folded pad 10a, folded as described above, be placed within the package with a main fold nose 214 disposed closest the package opening or top of the package (when the package is in an opened configuration) as suggested in FIG. 8. Placing a main fold nose 214 relative the package opening in this orientation presents the user with a single edge of the folded product that she can quickly and easily visually and/or tactilely identify, for easy and quick grasping of the entire pad, e.g., between a thumb and forefinger, and removal from the package. By contrast, when multiple edges of the folded pad are presented in the opening, user effort grasping and removing the pad from the package may require more slightly more concentration and/or effort. It has been discovered that users substantially prefer the former configuration, for this reason, and also for the reason that users perceive that this configuration of the pad within the package makes the pad more impervious to contamination.

Another feature that may be desired to improve user convenience is to impart contrasting colors or other visible characteristics to the respective outside visible surfaces of the folded pad 10a and the inside surfaces of the package material, thereby providing a visual contrast between the inside surfaces of the opened package and an outer surface of the pad as contained therein, visible immediately after the package is opened. This feature helps the user quickly visually identify the pad within the package for grasping and removal. Visual contrast may be imparted by any suitable techniques including tinting, pigmenting or printing the materials of the pad backsheet, the release film or paper, the package material, or any combination thereof. Herein, a “visual contrast” between colors or shades of two respective materials means that an ordinary observer having 20/20 vision (natural or corrected) and no substantial color vision deficiency, in normal office lighting conditions appropriate for desktop work, can perceive a contrast between the color(s) on the outside of the folded pad in the package, and the color(s) of the inside of the package material, with the package in an opened condition. Alternatively, for relatively close colors or shades, a “visual contrast” is identified when the value of delta E* determined through the Visual Contrast method below is equal to or greater than 2.0. For enhanced visual contrast, it may be preferred that the value of delta E* be equal to or greater than 3.5. Alternatively, where decorative designs are visibly present on one or both the outside of the folded pad as contained in the package, and the inside of the package material with the package in opened configuration, a “visual contrast” is identified when a design is visibly present on one but not the other, or when designs are visibly present on each but the designs differ in any visible respect.

### Process

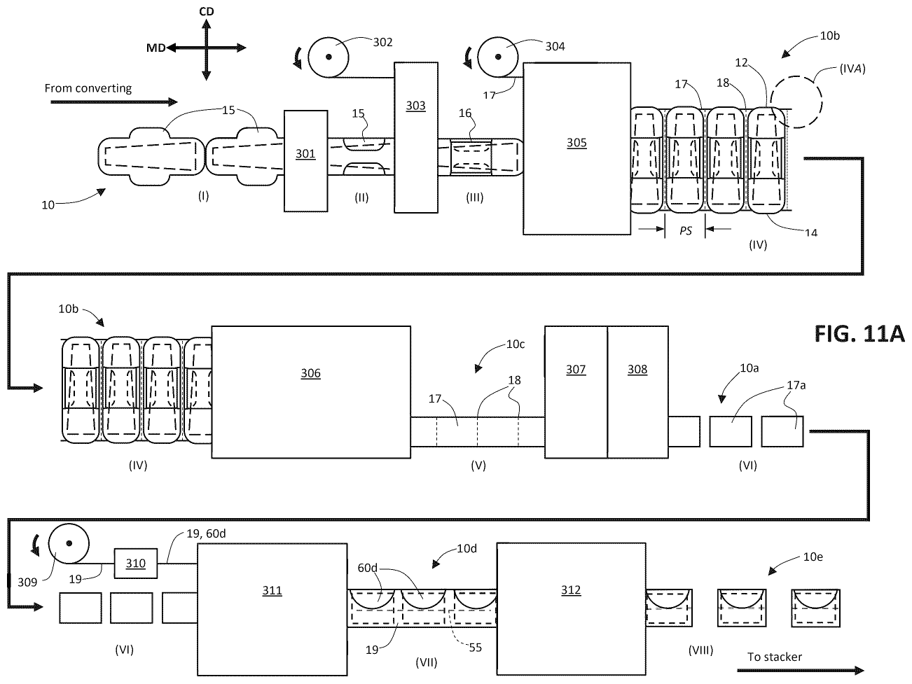

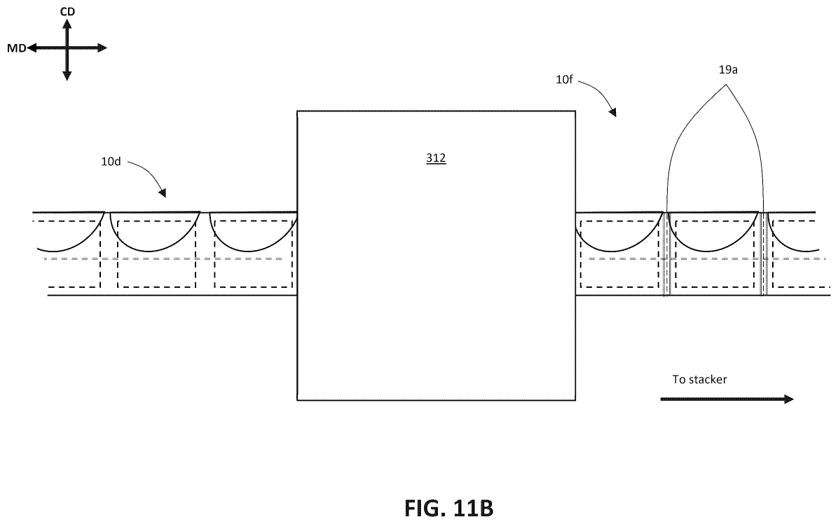

It has been learned that one or more of the process steps described below may be advantageous for imparting product features described above, and may provide additional advantages in manufacturing efficiency, reliability and quality assurance. Referring now to FIG. 11A, a process for manufacturing a packaged, folded feminine hygiene article is schematically depicted.

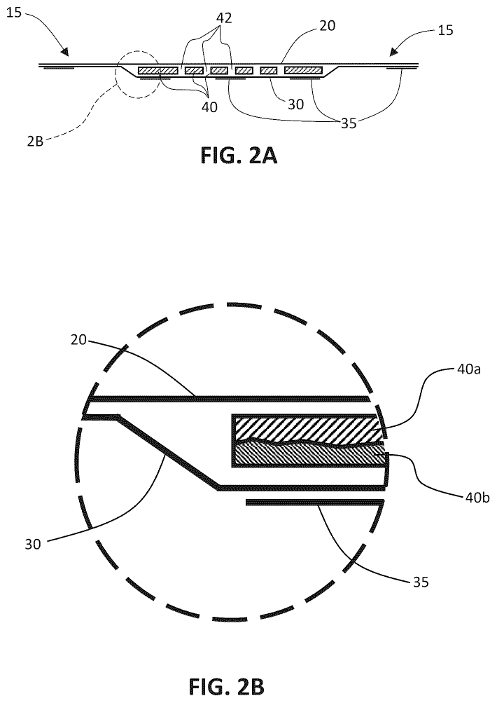

Starting at the top left of the figure, feminine hygiene pads 10 may be received from a converting line in configuration (I) in substantially finished condition, and with their longitudinal axes oriented in the machine direction MD. At the beginning of the process, pads 10 may have yet to have had backsheet adhesive 35 (see FIGS. 2A, 2B and 3) applied. Pads 10 may include wings 15, as previously described. Pads 10 may be conveyed from the converting line to a wing folding unit 301, which folds wings 15 (along, e.g., side longitudinal fold lines 204, see FIG. 1) over the wearer-facing/topsheet surfaces of the pads 10, from which they emerge in configuration (II) with the wings 15 folded over. (A more detailed example of a depiction of a pad as it might appear in configuration (II) appears in FIG. 3.)

In configuration (II), the pads may be conveyed to a wings coversheet application unit 303. The wings coversheet application unit 303 may be configured to receive wings coversheet stock from a supply roll 302 (which may be a roll of polymeric film or paper bearing an adhesive release surface or coating), cut it into individual wings coversheets 16, apply deposits of backsheet adhesive thereto, and place the individual wings coversheets 16 bearing the adhesive over the folded wings 15. Upon contact between the wings coversheet 16 and the folded over, outward-facing surfaces of the wings, the adhesive applied to the wings coversheet 16 can adhere and effectively transfer to the wings, so as to become a first portion of backsheet adhesive 35 deposited on the wings (see, e.g., FIGS. 2A and 3) and later remain in place on the wings when a user peels away the coversheet 16 in preparation to apply the pad to underwear. Prior to use, the wings coversheet 16 may serve to cover the adhesive and prevent it from being contaminated or adhering to surfaces not intended by the user. In some examples, the process may be configured to apply the adhesive directly to the wings directly/initially, rather than to the wings coversheets 16. The process can be configured such that the pads emerge from wings coversheet application unit 303 in configuration (III).

In configuration (III), the pads may be conveyed to a turn and re-pitch unit 305. Unit 305 may be a single unit, or a combination of cooperating units configured to receive individual pads moving with their longitudinal axes oriented in the machine direction MD, rotate them 90 degrees such that their longitudinal axes are oriented in the cross direction CD, phase them (i.e., space them along the MD by a predetermined distance), and place them onto a continuous web of backsheet adhesive coversheet stock 17. Unit 305 may also be configured to receive backsheet adhesive coversheet stock 17 from a supply roll 304 and place the rotated pads thereonto. Unit 305 may also be configured to apply a deposit of backsheet adhesive, either directly to the pads, or indirectly to the backsheet adhesive coversheet 17 stock, such that the adhesive applied to the backsheet coversheets can adhere and effectively transfer to outward-facing surfaces of the pads (e.g., the outward-facing surfaces of the pad backsheets), so as to become a second portion of backsheet adhesive 35 deposited on the pads (see, e.g., FIGS. 2A and 3) and later remain in place on the pads when a user peels away the backsheet adhesive coversheet 17 in preparation to apply the pad to underwear.

Backsheet adhesive coversheet stock 17 may be any polymeric film or paper material suitable for serving as a releasable coversheet for covering areas of the pad bearing backsheet adhesive 35. Further, in many circumstances, it may be desired that coversheet stock 17 be a material that is quiet, i.e., does not generate substantial audible noise, when manipulated by a user (such as when she removes the backsheet adhesive coversheet 17a from the pad in preparation to apply the pad to underwear). For this reason, a relatively quiet polymeric film may be selected for the coversheet stock 17. In some examples the film may be predominately PE, of an average caliper no greater than 0.08 mm (80 μm), preferably no greater than 0.065 mm (65 μm) and even more preferably no greater than 0.05 mm (50 μm). PE-based films tend to be relatively quiet as compared with paper, and with films based on other polymers. Other relatively quiet film materials might also be selected.

Backsheet adhesive coversheet stock 17 may be provided on the supply roll 304 with machine-direction-spaced separation perforations 18 already present, or alternatively, unit 305 may include a perforation unit (not specifically shown) that provides successive cross-direction lines of separation perforations 18 (or other suitable lines of weakness, such as scoring partially through the thickness of the backsheet adhesive coversheet stock), substantially uniformly spaced along the machine direction by distances approximately corresponding to the widths of the pads with wings folded. Spacing PS of separation perforations 18 along the coversheet stock 17 may include any desired margins beyond the pad widths, but in some circumstances, it may be desired that such margins be minimized for purposes of compactness and neatness of the completed folded pad product. Thus, in some examples it may be desired that machine-direction spacing PS of separation perforations 18 be no greater than 125 percent of the average width of the pads (wings, if present, folded), more preferably no greater than 120 percent of the average width of the pads, and even more preferably no greater than 115 percent of the average width of the pads. As a result, following completion according to the further process steps described below, the widths of the backsheet adhesive coversheets on the pad products will be, respectively, no greater than 125 percent of the average width of the pads (wings, if present, folded), more preferably no greater than 120 percent of the average width of the pads, and even more preferably no greater than 115 percent of the average width of the pads. It will be appreciated, however, that as a feature or result of the process steps described herein, in no case will the machine-direction spacing PS be less than 100 percent of the average width of the pads.

Additionally, in FIG. 11A, it can be observed that the individually oriented, spaced series of folded pads 10b in configuration (IV) may have, between them prior to folding, separation perforations 18 in the backsheet adhesive coversheet stock 17. It may be appreciated that the above-described method and any variant thereof, that successively places pads 10 on backsheet coversheet stock with the pads' longitudinal axes oriented in the cross direction CD, enables the manufacturer to provide coversheet stock 17 having a cross-direction dimension that is less than the length (LP, see FIG. 3) of the pads. To illustrate, referring to location (IVA) of FIG. 11A, it can be seen that coversheet stock 17 does not extend in the cross direction CD to the ends 12, 14 of the pads, but rather, lies short of them. This feature of the process is another way to enable the manufacturer to provide for a smaller, neater folded pad configuration because no unused margins of coversheet stock material 17 extend past the ends 12, 14 of the pads.

In configuration (IV), the series of folded pads 10b with adhered backsheet adhesive coversheet stock 17 may be conveyed to a folding unit 306. Folding unit 306 may be configured to continuously fold the series of folded pads 10b into two, three, four or more sections (for example, along fold lines 201, 202, 203, 204; see FIGS. 3, 4A and 4B and discussion above), resulting in a connected series of folded pads 10c (adhered to backsheet adhesive coversheet stock 17), in configuration (V). Folding unit 306 may be configured such that in configuration (V), the connected series of folded pads 10c are imparted with, for example, one of the folding configurations schematically illustrated in FIGS. 4A and 4B.

In configuration (V), the connected series of folded pads 10c may be conveyed to a separation unit 307. Separation unit 307 may be configured to continuously and successively exert machine direction tensile force on the successive pads, perpendicular to and across the lines of the separation perforations 18, causing the backsheet adhesive coversheet stock 17 to separate along the perforations 18. This results in a series of separated, folded pads 10a, each of which includes a backsheet adhesive coversheet 17a. Following separation, the folded, separated pads may be conveyed to a packaging phasing unit 308 configured to space the folded pads apart from one another along the machine direction, by a predetermined distance suitably selected for individual packaging as will be described below.

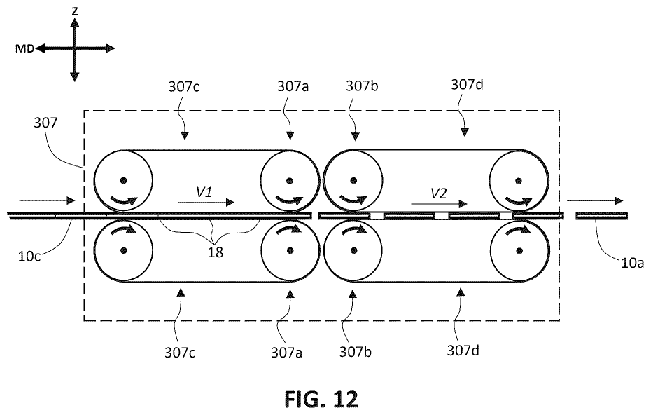

In some examples, referring to FIG. 12, separation unit 307 may include two successive pairs of opposing separation rollers including an upstream pair 307a and a downstream pair 307b. Each respective pair of separation rollers 307a, 307b may be configured to operate without conveyor belts (whereby the pads 10c, 10a pass directly through the nip between each pair of rollers), or with respective corresponding, respective upstream and downstream pairs of upper and lower conveyor belts 307c, 307d cycling about each of the rollers (whereby the pads pass between the respective upper and lower belts). Upstream separation roller pair 307a may be operated such that their circumferential roller surfaces move at a tangential/linear velocity V1. Downstream separation roller pair 307b may be operated such that their circumferential roller surfaces move at a tangential/linear velocity V2. To effect separation of successive pads, the system may be configured and operated such that V2 is greater than V1. As each connected pad in the series 10c emerges from the nip or space between pair 307a and enters the nip or space between pair 307b moving at a greater velocity, friction between the pad and pair 307b, or between the pad and belts 307d, increases machine direction tension in the pad between roller pairs 307a, 307b, and causes each, leading, exiting folded pad 10a to separate from the incoming/trailing series of pads 10c, along the separation perforations 18.

From the foregoing description it will be appreciated that the average width of the backsheet adhesive coversheets 17a on the separated folded pads 10a will be approximately equal to the spacing PS between the separation perforations 18 as shown in FIG. 11A. Thus, according to the description above, it may be desired that average width of the adhesive coversheets 17a be less than 125 percent, more preferably less than 120 percent, and even more preferably less than 115 percent (and in all cases at least 100 percent) of the width of the folded pads. For purposes of identifying presence of this feature and eliminating uncertainty resulting from variance in wing portion folding locations, the width of a folded pad is the greatest width of any portion of the absorbent layer 40 or absorbent core component disposed between the topsheet and backsheet. For purposes herein, averages may be calculated after measurements of adhesive backsheet coversheets and absorbent layer/core components of 10 examples of the product in question.

In view of the foregoing process description, the configuration of the separation perforations 18 may be important to efficient operation of the process as well as appearance of the resulting product. It may be desired that the separation perforations 18 through the coversheet stock 17 are not so extensive as to unacceptably compromise the tensile strength of the coversheet stock 17, i.e., its ability to withstand ordinary machine direction operating tension in the line upstream of the separation unit 307. If the separation perforations 18 are too extensive, the coversheet stock 17 may be vulnerable to premature, unwanted separation in processes upstream of the separation unit 307, which can necessitate line shutdown. On the other hand, it may be desired that the coversheet stock separate easily, neatly and cleanly in separation unit 307, so as to provide for smooth, even and uninterrupted conveyance of the pads through the separation unit 307 and the remainder of the line downstream, and provide a neat, finished appearance to the separated side edges of the individual backsheet adhesive coversheets 17a, such that the edges have a relatively smooth, not jagged appearance to the naked eye.

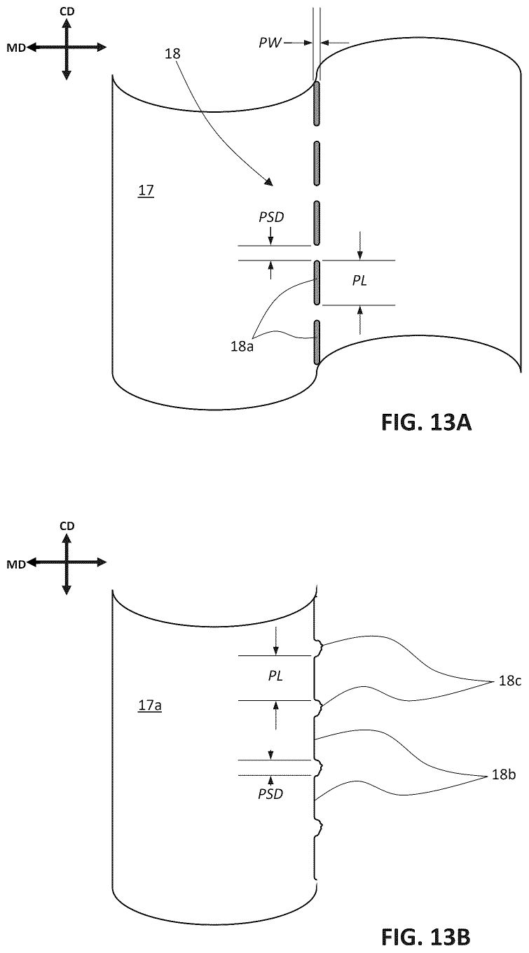

Referring to FIG. 13A, separation perforations 18 may be substantially uniformly dimensioned and spaced to provide for smooth and even separation. The perforations may be created mechanically, e.g., by use of a perforating die, or thermally, e.g., by use of a suitably selected and tuned laser. The perforations may penetrate the film entirely, or only partially, to an extent sufficient to propagate an orderly separation of the file along the perforations. Separation perforations 18 have an average length

PL in the cross direction CD, an average separation distance PSD in the cross direction CD, and an average perforation width PW in the machine direction MD. For purposes of balancing the needs for preserving suitable tensile strength in coversheet stock 17 upstream of the separation unit 307, providing for efficient separation of pads in separation unit 307, and providing for backsheet adhesive coversheets 17a with neatly separated edges in the finished product, it may be desired to configure perforating equipment such that perforations 18 have the following ranges of dimensions and ratio:
average PL=0.4 mm to 3.5 mm; more preferably 0.45 mm to 2.0 mm; and even more preferably 0.5 mm to 1.0 mm;
average PSD=0.7 mm to 2.5 mm; more preferably 0.75 mm to 1.8 mm; and even more preferably 0.80 mm to 1.1 mm;
average PW=0.015 to 0.1 mm; and
average PL/(average PL+average PSD)=0.15 to 0.70; more preferably 0.25 to 0.55; and even more preferably 0.30 to 0.50.

It will be appreciated that a sufficiently magnified plan view of separated side edges of backsheet adhesive coversheets 17a will approximately reflect such dimensions and ratio. Referring to FIG. 13B, a backsheet adhesive coversheet 17a processed according to the steps described herein (including such perforation step) will have a side edge oriented in the cross direction, having an appearance with features having similarities to those shown in FIG. 13B. There will be relatively neat edges 18b having cross direction dimensions approximately corresponding to length PL where the perforations were present (relatively easily identifiable and measurable with aid of suitable magnification equipment provided/combined with linear distance measuring aids), and tabs 18c where unperforated portions were present between the perforations, the distal ends of tabs 18c evidencing strain of the uncut portions of the film in the machine direction, to the point of failure of such portions of the film. The separation process imparts the tabs 18c with somewhat irregular lengths and irregularly-shaped distal end edges. Despite these irregularities, however, average lengths PSD and ratios average PL/(average PL+average PSD), or the two in combination, within the ranges set forth above, will result in relatively small and/or closely spaced tabs 18c that are not highly noticeable to the naked eye, retaining a neat appearance for the side edges of the backsheet adhesive coversheet 17a. For purposes herein of determining average values for PL and PSD for a given product, 10 examples of backsheet adhesive coversheets from 10 examples of the product in question are measured, each along a randomly-selected sample portion of the edge in question, 1.6 cm in length. Averages of the 10 measurements for each value are then calculated.

Following separation of the series of pads 10c into individual folded pads in separation unit 307, the individual folded pads may be conveyed to a packaging phasing unit 308, configured to receive the pads and reposition/space them apart along the machine direction by an average predetermined distance suitable for individual packaging described as follows. Suitably spaced, individual folded pads 10a then emerge from packaging phasing unit 308 in configuration (VI) as shown in FIG. 11A.

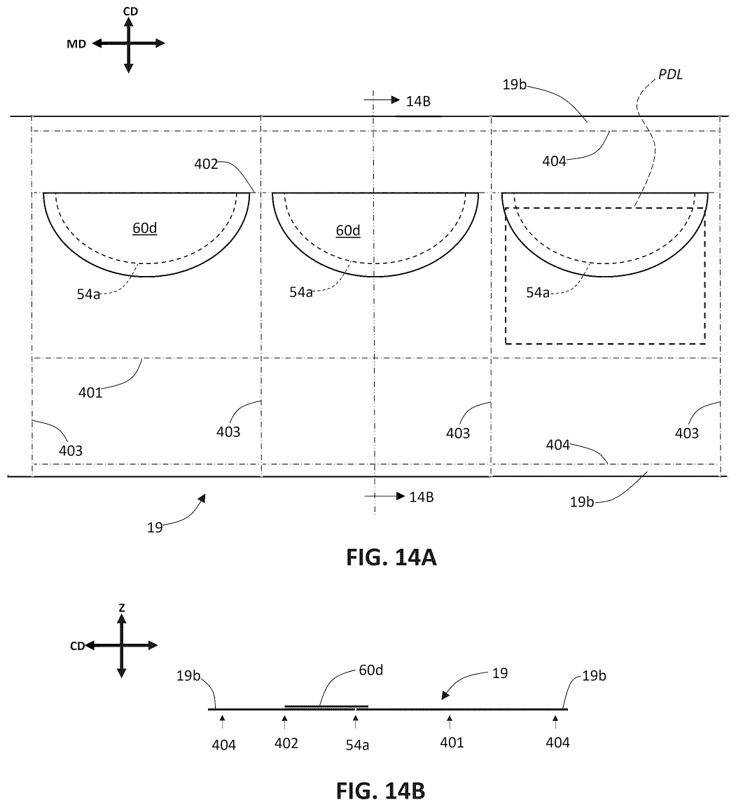

In configuration (VI), the individual folded, spaced pads 10a may be conveyed to a flow-wrap unit 311. Flow-wrap unit 311 may be configured to receive package film 19 from a package film supply roll 309. In some examples the package film may include PE, and have an average caliper no greater than 0.08 mm (80 μm), preferer ably no greater than 0.065 mm (65 μm) and even more preferably no greater than 0.05 mm (50 μm). Referring to FIGS. 14A and 14B in conjunction with FIG. 11A, package film 19 already bearing pre-applied flap stickers 60d overlying pre-formed package opening cuts or perforations 54a in the film 19, may be received by flow-wrap unit 311. Alternatively, package film 19 may be received already bearing pre-applied flap stickers, but the film may yet to have package opening cuts or perforations 54a provided; and flow-wrap unit 311 may include or be preceded by a package film process unit 310 that provides package opening cuts or perforations 54a in package film 19. Alternatively, package film process unit 310 may be configured to perform either or both of application of flap stickers 60d to the package film 19, and provision of package opening cuts or perforations 54a in the package film 19.

Package opening cuts or perforations 54a may be intermittent perforations or continuous cuts that penetrate substantially or all of the entire thickness/caliper of the package film 19 along the intended path of separation. A supplier of pre-cut package film, or the manufacturer of the product who performs such perforating or cutting, may be provided advantage by application of flap stickers 60d to the film prior to perforating or cutting. Perforating or cutting may be performed continuously using roller-based die-cutting equipment, and the presence of stickers 60d makes it unnecessary to adjust the equipment such that the die cutting blade edges contact an opposing anvil roller to effect a cut through substantially the entire caliper or thickness of the film. Rather, previously-placed flap stickers 60d on the film 19 can effectively provide relatively soft “anvil” surfaces against which the die cutting blades operate. Clearances in the cutting equipment may be adjusted such that the die-cutting blades substantially penetrate/cut through the film 19, but not the flap stickers. In this manner, die cutting blade life may be extended because the die-cutting blade edges are not required to contact a relatively hard surface of an opposing anvil roller.

A continuous cut 54a in the film along the intended opening edge 54, in contrast to intermittent perforations, may provide a benefit for the consumer/user in that no tearing of package film 19 (at uncut or unperforated portions between perforations) is required to open the package, thereby generating less noise by opening the package.

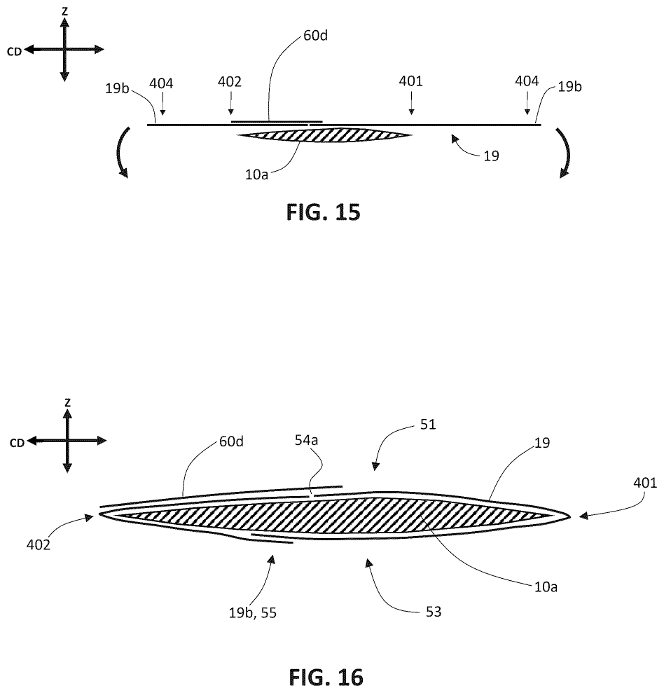

As it continuously intakes package film 19, flow-wrap unit 311 also intakes the spaced (phased), individual folded pads 10a and successively disposes them in contact with the film along pre-determined (phased) intervals wherein the pad machine direction spacing corresponds and is registered with the film such that each pad is substantially evenly disposed/aligned between side crimp/seal/cut lines along the cross direction CD of the film, and between package bottom and top fold lines 401 and 402. Such disposition is illustrated by way of example by pad disposition location PDL shown in FIG. 14A. Referring to FIGS. 15 and 16, flow-wrap unit 311 may be configured to then continuously wrap package film 19 over and about the succession of advancing pads 10a (e.g. via plow folding equipment). Referring again to FIG. 14A, package film 19 may be provided with seam margins 19b of extra film material extending (in the cross direction) outboard of seam margin lines 404, to provide material to create a continuous machine-direction seam 55 joining the machine-direction edges of the package film 19 after it is wrapped about the pads 10a. The flow-wrap unit 311 may include or be accompanied by equipment for forming the seams via use of adhesive, via thermal fusion of the package film, or a combination thereof, along the seam margins 19b. The seaming equipment may be configured to form a continuous machine-direction fin seam (such as shown in cross section, by way of example, in FIG. 10B (seam 55)), or alternatively, an overlap seam (such as shown in cross section, by way of example, in FIG. 16 (seam 55)). As previously noted in some examples it may be desired that attachment between the joined sections forming back panel 53, at seam 55, whether effected by adhesive or by welding/fusion of the film, be intermittent or discontinuous and may have spaced intervals of attachment, rather than being continuous, along seam 55. This may be desired to allow venting of air from within the finished package upon compression of the package, such as may occur in processes downstream of package formation, reducing chances that the package will burst open along seams or along the flap closure such as upon compression of the completed package.

Referring back to FIG. 11A, a continuous series of folded pads 10d flow wrapped along the machine direction, emerge from the flow-wrap unit 311 in configuration (VII), also depicted by way of example, in cross section, taken along the cross-direction, in FIG. 16. In configuration (VII), the series 10d may be conveyed to a package crimp sealing and cutting unit 312. Package crimp sealing and cutting unit 312 may be configured to continuously receive the series 10d and then, at intervals corresponding to side crimp/seal/cut lines 403 (see FIG. 14) crimp the package film 19 to form front and rear panels 51, 53 together, adhere or fuse the package film of the respective panels to form cross direction side seams 52 (illustrated in, e.g., FIGS. 5A and 9A) and cut the individual packages apart between successive trailing and leading portions of the seamed film material. Individually packaged pads 10e, in configuration (VIII), emerge from the package crimp sealing and cutting unit 312, and may then be conveyed to a stacker (not shown) for controlled collection, orderly stacking and further packaging of quantities of individual packages, as desired. Adhesion or fusion of the package film of the front and rear panels to form side seams 52, and/or cutting between successive leading and trailing packages, may be effected by direct, localized application of heat, ultrasonic energy or any other mechanism suitable to effect localized joining of the film of the front and rear panels. In some examples, seaming and cutting may occur simultaneously, via, e.g., a heated, appropriately shaped knife or die.

In some examples, rather than cutting the individual packages apart entirely as suggested in FIG. 11A, as suggested in FIG. 11B, unit 312 may be configured to create package separation perforations 19a between the formed seams of respective leading and trailing packages, as they pass through the unit, resulting in a continuous chain 10f of individual packages held together by the uncut portions of film within/along the paths of the package separation perforations between the respective package side seams of adjacent leading and trailing packages. This provides a chain of packages 10f that may be gathered by rolling, or folding or festooning with each other in, e.g., accordion-fashion, in a desired quantity, for a desired alternative mode of packaging and/or dispensation. Packages supplied in this mode may be separated from each other by tearing along the package separation perforations 19a.

It is also contemplated that processes by which individual pads are flow-wrapped and envelope packages are formed thereabout, described above, may be applied in other examples wherein the folded pads are conveyed into flow wrapping unit 311 with their longitudinal axes oriented in the machine direction MD, rather than in the cross direction CD as described above. The resulting individually packaged pad will appear in the package with one side edge exposed at the access opening when the package is opening, and the other side edge within the package proximate the opposite, closed end of the package.

For other alternative examples, a process for creating side seams 52, and separating the individual packages 50, is contemplated. A process whereby side seams 52 that are relatively narrow has been developed, which enables (1) rapid and efficient creation of narrow but secure seams; (2) relatively close clearances between the contained folded pad 10a and the inside lateral dimension of the envelope between the side seams, enabling (3) material savings and (4) a more compact package. The process contemplated herein forms narrow side seams and separates respective seamed leading and trailing packages simultaneously, with a minimized margin of package film material that is not utilized for enclosing space for the pad product contained within the envelope package.

Referring to FIGS. 11A and 17-21, unit 312 may be a package seaming and severing unit and may include an opposing pair of rotating rollers 313, 317, having seaming and severing elements that meet at a transitory nip 318b therebetween. The rollers are driven to rotate about respective axes 313a, 317a that are oriented in the cross direction CD. An incoming continuous series of flow-wrapped pads, spaced apart along the machine direction MD within the package film 19 wrapping by a desired spacing, is conveyed into the transitory nip 318b. At least one of the rollers 313, 317 is equipped with one or more seaming and severing elements such as, for example, seaming knives 318. The other roller also may be equipped with opposing seaming and severing elements such as, for example, anvils 314. The rollers are configured such that seaming knives meet seaming knives in a transitory nip (not shown), or alternatively (as shown), seaming knives 318 meet anvils 314 in the transitory nip 318b, as the rollers rotate. In some examples (not shown), roller 313 may have a simple cylindrical surface, portions of or the entirety of which serves as an anvil. The rollers 313 and 317 are configured, the process is controlled, and the machine direction spacing of folded pads 10a is phased, such that the seaming knives 318 meet opposing seaming knives or surface(s) of anvils 314 in the transitory nip 318b, and force respective portions of package film 19i and 19ii together, in spaces between the folded pads 10a. In some configurations (not shown), opposing seaming knives mounted on the respective rollers 313, 317 may meet each other. In the particular, non-limiting, exemplary configuration shown, knife roller 317 has seaming knives 318 mounted thereon, and anvil roller 313 has individual anvils 314 mounted thereon, whereby edges 318a of seaming knives 318 contact substantially flat or even planar anvil surfaces to effect seaming and separation of successive pairs of leading and trailing packages. Seaming knives 318 are configured on roller 317 such that their distal (severing) edges 318a extend along the cross direction.

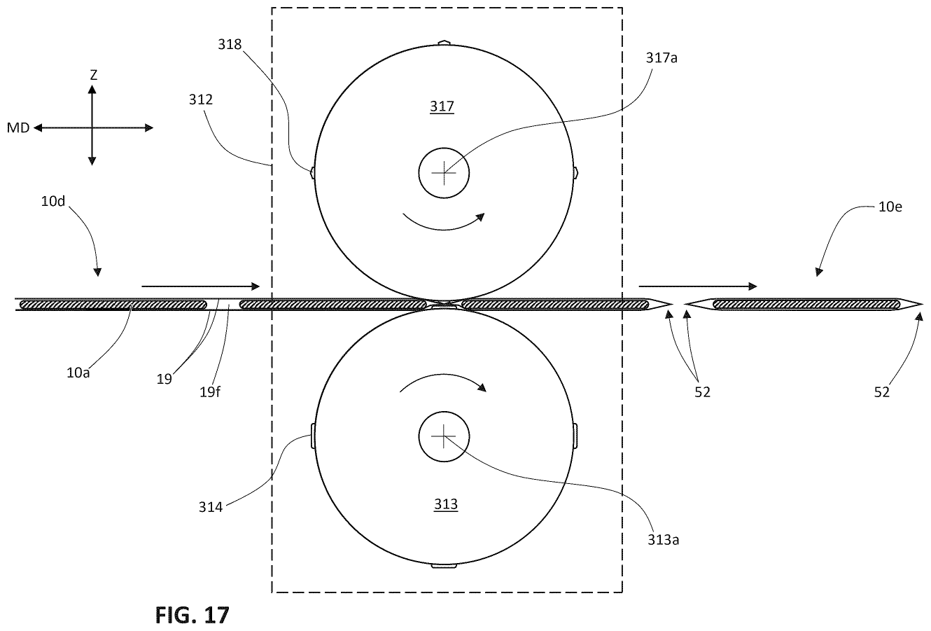

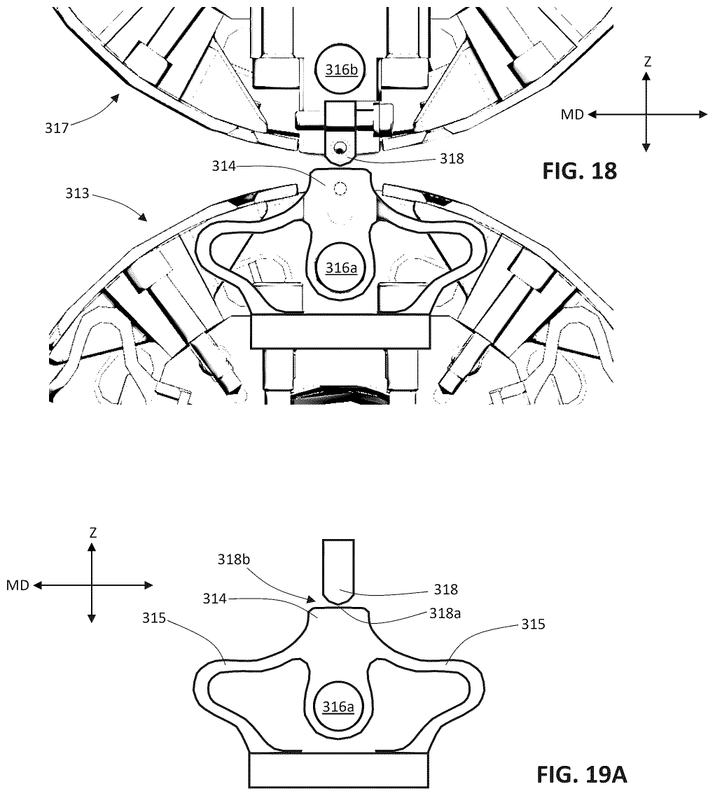

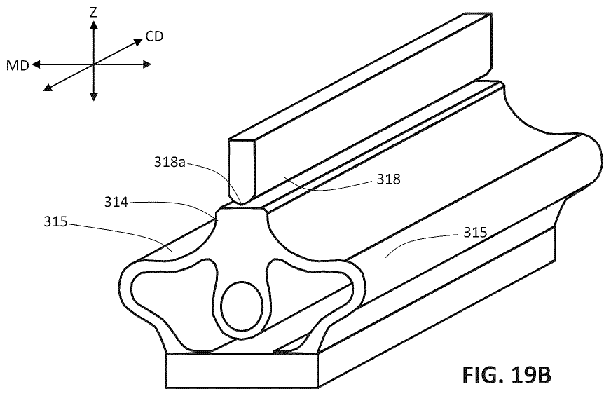

Referring to FIGS. 17 and 21, as the package film portions 19i and 19ii pass through the transitory nip 318b (with no pad between them, at the phased spaces 19f) they are compressed against each other with greatest concentration of pressure at the distal edges 318a of the seaming knives 318 (resulting from opposing force between seaming knives 318 and anvils 314), sufficient to cause material forming these portions to be rapidly compressed together, plastically deformed and expressed forwardly and rearwardly along the machine direction, away from the knife edges 318a, as schematically illustrated and indicated by the large arrows in FIG. 21. Clearance between the seaming knife edges 318a and the anvils 314 in the transitory nip 318b is, preferably, specified and maintained to be zero (or, when a pliant mechanism is included as described below, a negative clearance value), such that substantially all package film material is expressed at the transitory nip 318b, and substantial severing of successive leading and trailing packages is effected. At the same time, the rapid compression and plastic deformation of the polymeric film material of the package film 19 as portions 19i and 19ii are compressed against each other in, and plastically expressed from, the transitory nip 318b, causes wrinkling and deformation of material at the interface 52b between the film portions (as suggested in FIG. 21), and resulting physical entanglement or physical interlacing of the respective material portions along the interface. Without intending to be bound by theory, it is believed, further, that the rapid compression and forced plastic deformation of the material in the transitory nip 318b may also generate internal friction in the film material sufficient to cause some heating and contribute to effecting some fusion bonding between the portions 19i and 19ii, whereby the interface 52b between the materials becomes at least partially if not entirely indistinct. Without intending to be bound by theory, it is believed that this combination of physical entanglement or interlacing between the film portions along the interface, with some potential fusion bonding, are effective to create suitably strong attachment between the front and rear portions of the package film, and thus, effective side seams immediately proximate the line of severance between leading and trailing packages, effected by compression between the seaming knife 318 and anvil 314 in the transitory nip. Advantageously, this seaming and severing may be accomplished at relatively high throughput speeds. Referring to FIGS. 18 and 19A, in some examples, one or both of the seaming knives 318 and anvils 314, or surrounding proximate components of these parts or of the roller assemblies, may be supplied with heating energy, which may be transferred to the film 19 in the transitory nip and thereby increase likelihood of effecting at least some thermal fusion bonding (assuming that film 19 comprises thermoplastic(s)). For example, heating elements 316a and 316b may be incorporated, respectively, into the seaming knife, anvil and/or associated proximate components of the rollers 313, 317. One or both of the heating elements may be configured and controlled to maintain a temperature at the acting surface of the associated seaming and severing element sufficient to promote at least some fusion of thermoplastic material at the inside-facing surfaces of film 19.

Seaming elements such as knife 318 and anvil 314, or at least the portions thereof that meet in the transitory nip, are formed of suitable tool steel or other suitably hard, long-wearing and fracture-resistant material.

Any one or more of the seaming and severing elements such as seaming knife 318 and/or anvil 314 and/or cooperative structure of the roller(s) may be provided with a pliant mechanism, that enables one or both to elastically move radially toward their roller axes as radial force is exerted by each against the other at the transitory nip. In the example depicted in FIGS. 18, 19A and 19B, anvil 314 is supported by elastic pliant anvil supports 315, which are directly or indirectly mounted to the body of roller 313. Pliant anvil supports 315 may be spring members that are elastically flexible so as to permit anvil 314 to move radially toward the axis of roller 313, in response to radial force exerted against anvil 314 by seaming knife 318, when the two meet at the transitory nip. This pliant mechanism reduces or relieves any need for adjusting the rollers periodically to ensure zero clearance in the transitory nip, desired for good seam formation and clean severing. The pliant mechanism also reduces wear and contributes to extending the useful life of the seaming knife edge 318a and anvil 314 surface. Because it provides for radial displacement of the anvil and/or seaming knife it supports, the system may be configured with negative clearance in the transitory nip, i.e., a value that causes and requires such radial displacement during the time the seaming knife and anvil are in contact with each other and rotate through the transitory nip, in order for rotation to occur and proceed (i.e., not be prevented by interference between the seaming knife and anvil). In addition to the benefits described immediately above, such a configuration and radial displacement increases the time during which the seaming and severing elements, e.g., knife edge and anvil, are in acting opposition and compressing the film portions together (dwell time), through the transitory nip. If there were no radial displacement of either or both of the seaming knife and the anvil, dwell time would be limited to the very brief amount of time that the knife edge and anvil meet in a nip with zero clearance. Increasing dwell time via radial displacement of either or both the knife and anvil provides more time for, and thereby improves the process of, material interlacing and potential fusion bonding, as the material moves through the transitory nip. Thus, increasing dwell time in this manner enables seaming and severing at higher throughput speeds.

The pliant mechanism such as pliant anvil supports 315 may be configured and manufactured to have any spring constant required to exert a desired amount of force opposing the seaming knife 318 at a selected radial displacement of the anvil in the transitory nip, effective to cause nearly or substantially complete expression of film material from the transitory nip, while not causing excessive resistance to rotation through the transitory nip.

Using the process and equipment described above, it is possible to simultaneously form narrow/thin but secure package seams 52, and sever successive leading and trailing packages, in a single process step, at a relatively high throughput rate. Characteristically, no margin of extra film material is required to be present to the outsides of the seams relative the main portion of the package, remaining to be perforated or cut to separate individual packages, in a downstream process step. As an advantageous result, less film material is required to form a succession of individual packages. Substantially no, or no, adhesive material is required to be added to the film 19 to effect the seams 52, and so, characteristically, a majority or substantially an entirety of the lengths of seams 52 may include no added adhesive material.

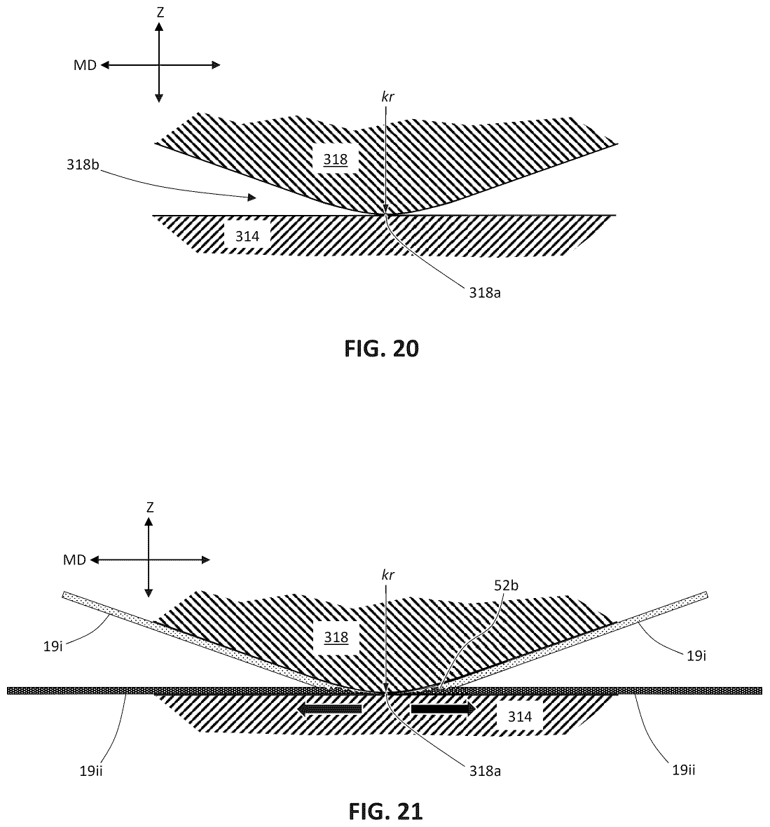

Referring to FIG. 20, it has been learned that the value selected for radius kr of the knife edge 318a (imparted during manufacturing thereof) may be important to ensuring successful formation of seams and effective severing of leading from trailing packages at the relatively high throughput rates contemplated herein. This value is selected so as to meet the objectives of effecting attachment between film portions 19i and 19ii, and severing the respective portions of the package film 19 in a manner that forms edge seams 52 that are relatively narrow, but still acceptably strong and resistant to failure during handling in downstream processes, and during carry and handling by the consumer prior to intentional opening of the package. The edge radius kr of the knife edge is the radius of the knife edge as measured in a plane defined by the machine and Z-directions, that transitions from the forward surface(s) of the knife to the rearward surface(s) of the knife, and contacts the anvil surface. It will be noted from the description above that the purpose is to compress the film portions together and cause plastic deformation and flow of the film material thereof away from the transitory nip in forward and rearward directions (along the machine direction MD), to simultaneously effect formation of seams and separation, rather than to shear the film material. The edge radius kr should be selected so as to provide a region of compression of the material, that tapers off relatively quickly moving away from the distal-most portion of knife edge 318a and the line of separation of the packages. It has been learned that the desired edge radius kr should be from 2 mm to 5 mm, when an opposing anvil has a substantially flat or planar surface (i.e., radius of any curvature of acting surface equal to or greater than the outermost radius of its path of rotation about its roller axis), for a package film 19 having a caliper, in non-limiting examples, of 20 μm to 60 μm.

In other examples, noted above (but not shown), the rollers may be configured such that two fixtures having substantially radiused acting edges, e.g., two seaming knives 318 or other seaming and severing elements (one on each roller), for each seaming and severance action, oppose each other in a transitory nip, rather than one seaming knife 318 and one anvil 314 (where an “anvil” has a substantially flat or planar acting surface (i.e., radius of any curvature of acting surface is equal to or greater than the outermost radius of its path of rotation about its roller axis) opposing a knife edge. It would remain that the seaming and severing elements would meet in the transitory nip along an identifiable line of contact therebetween, extending along the cross direction, parallel to the axes of the rollers. In such examples, the edge radius for each of the respective opposing seaming knives or other seaming and severing elements would be adjusted to have the desired effect of rapid formation of thin but secure seams via compression and displacement of film material in the transitory nip.

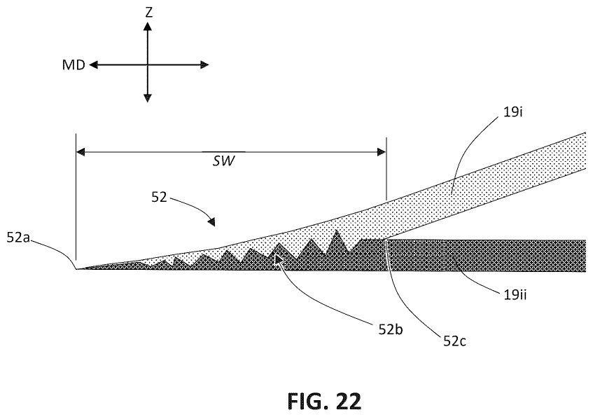

It is believed that opposing seaming and severing elements (such, for example, a knife and anvil) having these geometric values/dimensions, in combination with at least one pliant structure supporting one or both elements, will be effective to simultaneously effect severance of respective leading and trailing packages, and form acceptably strong but desirably narrow edge seams holding the respective film portions together, on either side of the line of severance, at a relatively high throughput rate. Minimizing side seam width enables use of less material to successively create individual envelope packages, and enables creation of smaller (i.e., desirably, more compact) packages for the same product (e.g., a folded feminine hygiene pad). Referring to FIGS. 22 and 25, it is possible to effect suitably strong seams 52 having an average seam width SW (illustrated in FIG. 25) in the machine direction MD of 200μm to 1,000 μm, or 200 μm to 800 μm, or 300 μm to 600 μm, or within these ranges, equal to or less than 500 μm. Referring to FIG. 26, in the context of packaging folded feminine hygiene pads as described herein, it is possible to utilize the described seaming and severing process and apparatus described herein in which the difference between folded pad width PD and inside package enveloped width EW (measured in any suitable manner, with the pad outside of the package and the package lying flat) is equal to greater than 4 mm but no greater than 25 mm, preferably no greater than 20 mm, and more preferably no greater than 15 mm.

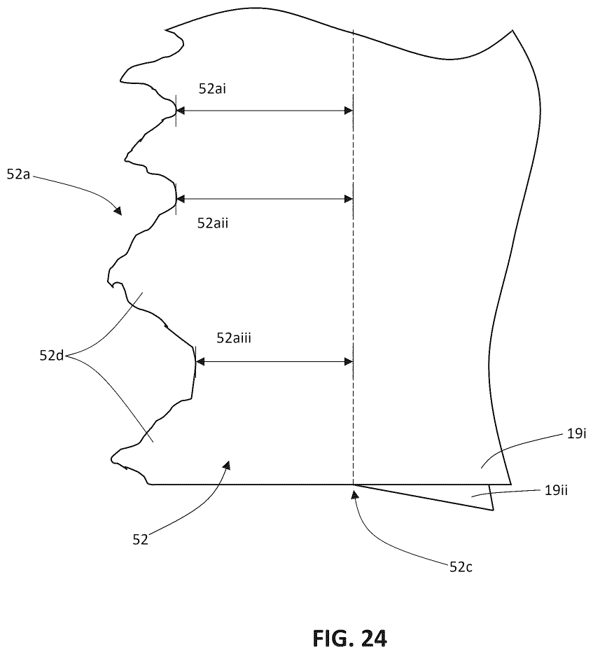

Referring to FIG. 24, seam width is measured between the location 52c at which affixation between the film portions begins on the inside of the seam, and the outside severed edge 52a, at innermost locations between separation tongues 52d, e.g., locations 52ai, 52aii and 52aiii. Separation tongues 52d along severed edges 52a are characteristic of the process described above, but are microscopic in size and typically not substantially noticeable to the human eye without aid of magnification. Without intending to be bound by theory, it is believed that they occur as a result of microscopic imperfections in the surfaces of the knife edge 318a and anvil 314, where they meet at the transitory nip. These microscopic imperfections can prevent perfect continuous contact between the knife edge and the anvil in the nip, and thereby prevent film 19 material from being completely expressed in the nip, leaving a microscopically thin film of irregular caliper, constituted by the material forming film portions 19i and 19ii, connecting leading and trailing packages emerging from the nip. This microscopically thin film is easily fractured and separated via application of slight machine direction tension in any suitable downstream process including conveying, leaving behind separation tongues 52d, which impart a microscopically irregular profile to severed edge 52a. The separation tongues are the result of plastic deformation and strain until fracture of the irregular, microscopically thin film between the seams emerging from the nip.

Persons of ordinary skill in the art will be able to determine a suitable method of measuring and calculating an average seam width based upon randomly sampling at least 5 locations along the seam length and cutting the material along an MD-Z-direction plane, aided by magnification techniques, as reflected, for example, in FIG. 24 (which reflects a photo and a 100 μm scale at lower right). The appropriate location for making sample seam width measurements is at the “valleys” between separation tongues 52d, as illustrated in FIG. 25 (e.g., “valley” locations 52ai, 52aii, 52aiii).

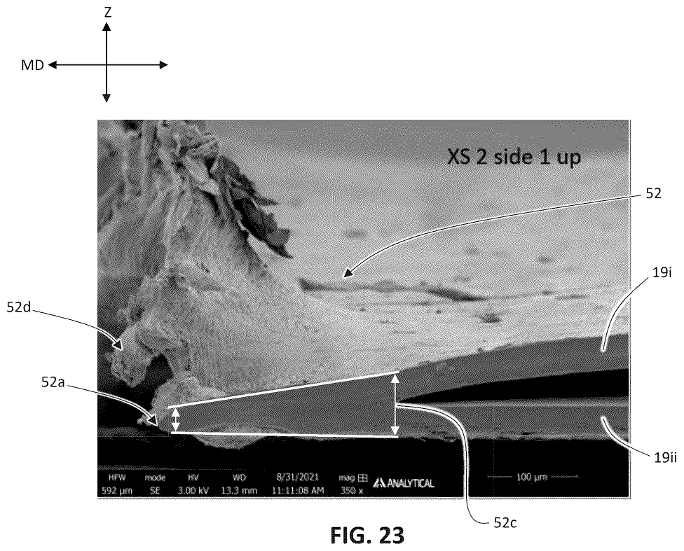

As may be appreciated from FIGS. 22 and 23, another characteristic of the seam resulting from the process contemplated herein, is that the package caliper (with respect to FIGS. 22 and 23, measured along the Z-direction) decreases or tapers down, from the location 52c where the inside portion of the seam begins, to the severed edge 52a. This is a result of the geometry of the tooling, described above, and the seam formation process.

The selection of material(s) to constitute the package film 19 to be seamed and severed via the process and equipment described above may be important. The package film 19 may be formed substantially of only a single layer of material. It also may be formed of two or more layers, the materials of which may be selected and included for differing purposes including but not limited to fusibility, ductility, adding tensile strength, providing anisotropic tensile strength, providing other mechanical properties, providing relative quietness when manipulated (e.g., consumers may prefer a package film that does not generate a noticeable “crinkling” noise during handling and opening of the package), compatibility with desired printing inks and/or amenability to printing or other techniques for imprinting text or visual artwork, cost, etc.

For purposes of the facilitating the process of seaming and separation described above, however, materials of package film 19 and portions 19i and 19ii should include a thermoplastic polymer material, at least, in the layer(s) forming the inward-facing surfaces (the surfaces that will meet and form an interface 52b in the transitory nip between rollers 313, 317). It may be preferable that the selected thermoplastic polymer material have relatively high ductility (for enabling plastic deformation along/about interface 52b (see FIG. 21)) and/or a low melting temperature relative that of other layer(s), to maximize potential for physical engagement/interlacing and potential fusion bonding between the film portions during the seaming and severing process.

At the same time, a thermoplastic polymer material having relatively high ductility and a relatively low melting temperature may lack a desirable level of tensile strength desired for the package, at commercially feasible film basis weights for products of the type contemplated herein. Such material may also be more likely to form larger separation tongues 52d (see FIGS. 23 and 24). In order to supplement tensile strength and reduce formation of separation tongues, a multi-layer package film having differing layer components may be used as package film 19. Referring to a non-limiting example illustrated in FIG. 28, an inside-facing layer 19c, a second layer 19d and a third layer 19e are shown. Inside-facing layer 19c is the layer that will forming the inside surfaces of the envelope package and will meet and be forced against an opposing film portion in the transitory nip of the seaming and severing rollers. Thus, it may be preferred that this layer 19c have relatively high ductility and/or relatively low melting point, relative these properties of the other layer(s). Material(s) forming second layer 19d and/or third layer 19e (if included) may be chosen for having differing properties, e.g., greater tensile strength, greater brittleness and/or relatively higher melting temperature, than that of layer 19c. A material having greater brittleness and/or relatively higher melting temperature, than that of layer 19c may cause the entirety of the film 19 to be less susceptible to formation of large separation tongues 52d. Material to constitute the second and/or third layers 19d, 19e may also be chosen for imparting greater tensile strength per unit basis weight than the material constituting inside-facing layer 19c.

Thus, in some examples, an inside-facing layer 19c may be constituted, preferably in weight majority, by a PE, such as a low density polyethylene (LDPE) such as linear-low density polyethylene (LLDPE), and a second layer 19d may include a polypropylene. In a particular non-limiting example, the inside-facing layer 19c may include a metallocene-based polyethylene, and in some examples, a majority weight percentage of metallocene-based polyethylene. In the process described herein, a metallocene-based polyethylene such as metallocene-based LLDPE (m-LLDPE) in the inside-facing layer may contribute to formation of stronger seams. The second layer 19d may include any suitable weight percentage of polypropylene. It has been learned that inclusion of polypropylene in a layer of film 19, such as second layer 19d, helps reduce the formation and/or size of any separation tongues 52d. In some non-limiting examples, inside-facing layer 19c may be constituted of from 20 percent to 40 percent by weight polypropylene, in addition to any of the polyethylenes noted above. In some non-limiting examples, second layer 19d may be constituted of from 20 percent to 40 percent by weight polypropylene, in addition to any of the polyethylenes noted above. In the second layer, it may be preferred that polypropylene constitute no more than 30 weight percent, preferably no more than 25 weight percent, and even more preferably no more than 22 weight percent of the second layer. Without intending to be bound by theory, it is believed that inclusion of polypropylene in either the first or second layers greater than the upper ends of the ranges set forth above may have a deleterious effect on seam strength and/or severed edge quality. However, addition of polypropylene as a component of one or more layer contributes to imparting tensile strength to the film, beyond that imparted by polyethylene alone.

In some examples, a third layer 19e may be desirable, which may be an outward-facing layer. Material constituting a third layer 19e may be chosen for properties that include compatibility with printing inks (where printing is to be included on the package), a pleasant and/or smooth and/or non-tacky feel and/or low noise generation during handling and manipulation by the consumer (for discreetness). Polyethylenes are believed to be useful in this latter regard. In some non-limiting examples, a third layer 19e may be included and may be constituted of LDPE or linear low density polyethylene (LLDPE).

A multilayer film can be produced with cast extrusion or blown extrusion equipment, provided that the equipment used is configured to be capable of producing films within the range of calipers described herein. Without intending to be bound by theory, however, it is believed that producing films having characteristics suitable for purposes contemplated herein may be more efficiently produced using a blown film process. In a blown film process, the molten, extruded polymer film components are stretched in multiple directions after they emerge from the forming die. As a result, after cooling and stabilizing, the blown film may have tensile strength that is less isotropic. This may enhance strength and robustness of the formed seams as described herein, as well as of the package generally, such that a lower basis weight (and thus, lower cost) package film 19 may be used. A multilayer film may be coextruded and blown.

### Examples Contemplated

In view of the foregoing disclosure, the following non-limiting examples are contemplated:

### A. Packaged Product

A1. A packaged feminine hygiene pad product, comprising:
an envelope package formed of polymeric film, the package comprising a pair of substantially straight parallel opposing side seams at which a first portion of the film is permanently affixed to a second portion of the film to form an affixed region, the respective affixed regions defining an enveloped width EW, measured along a direction perpendicular to the side seams, of enveloped space between the side seams;
a feminine hygiene pad laterally folded over on itself along at least three lateral fold lines, and disposed within the enveloped space, wherein the pad as folded has a folded pad dimension PD, measured along a direction parallel to the enveloped width EW;
wherein the value (EW-PD) is equal to or greater than 4 mm, but equal to or less than 25 mm, preferably 20 mm, and more preferably 16 mm.

A2. The product of example A1 wherein the affixed regions have a maximum average seam width SW along the direction of the inner dimension, of from 200 μm to 1,000 μm, or from 200 μm to 800 μm, or from 300 μm to 600 μm, or within these ranges, equal to or less than 500 μm.

A3. The product of either of the preceding examples wherein the respective affixed regions are severed at side edges (52a) of the envelope package, with no unaffixed margins of the first and second film portions together extending laterally beyond the side seams.

A4. The product of example A3 wherein the envelope package tapers down in caliper from a location at an inboard edge of an affixed region, outwardly along the envelope width direction, to a location at a side edge.

A5. The product of any of the preceding examples wherein some, preferably a majority, and more preferably substantially all of the respective affixed regions are affixed together without added adhesive material.

A6. The product of any of the preceding examples wherein the polymeric film comprises blown film.

A7. The product of any of the preceding examples wherein the polymeric film comprises at least an inward-facing layer and a second layer.

A8. The product of any of the preceding examples wherein the polymeric film comprises an inward-facing layer comprising PE, preferably LDPE, more preferably LLDPE, and even more preferably m-LLDPE.

A9. The product of either of examples A7 or A8 wherein the polymeric film comprises an inward-facing layer comprising PE, preferably LDPE, more preferably LLDPE, and even more preferably m-LLDPE, and at least a second layer comprising polypropylene.

A10. The product of example A9 wherein the polypropylene constitutes 20 to 40 weight percent of the second layer, and preferably no greater than 30 weight percent, more preferably no greater than 25 weight percent, and even more preferably no greater than 22 weight percent.

A11. The product of any of examples A7-A10 wherein the second layer constitutes 50 percent, preferably 55 percent or more, of a total basis weight of the film.

A12. A packaged feminine hygiene pad product, comprising:
an envelope package formed of polymeric film, the package comprising a pair of substantially straight parallel opposing side seams at which a first portion of the film is permanently affixed to a second portion of the film to form an affixed region, the respective affixed regions defining an enveloped width EW, measured along a direction perpendicular to the side seams (52), of enveloped space between the side seams;

a feminine hygiene pad laterally folded over on itself along at least three lateral fold lines, and disposed within the enveloped space, wherein the pad as folded has a folded pad dimension PD, measured along a direction parallel to the enveloped width EW;

wherein the affixed regions each have an average width SW along the direction of the enveloped width EW, of 200 μm to 1,000 μm, preferably 200 μm to 800 μm, more preferably 300 μm to 600 μm, or within these ranges, even more preferably equal to or less than 500 μm;

wherein the respective affixed regions are severed to form side edges of the envelope package; and

wherein the envelope package tapers down in caliper from a location at an inboard edge of an affixed region, outwardly along the envelope width direction, to a location at a side edge.

A13. The product of any of the preceding examples wherein some, preferably a majority, and more preferably substantially all of the respective affixed regions are affixed together without added adhesive material.

A14. The product of either of examples A12 or A13 wherein the polymeric film comprises blown film.

A15. The product of any of examples A12-A14 wherein the polymeric film comprises at least an inward-facing layer and a second layer.

A16. The product of any of examples A12-A15 wherein the polymeric film comprises an inward-facing layer comprising PE, preferably LDPE, more preferably LLDPE, and even more preferably m-LLDPE.

A17. The product of either of examples A15 or A16 wherein the polymeric film comprises an inward-facing layer comprising PE, preferably LDPE, more preferably LLDPE, and even more preferably m-LLDPE, and at least a second layer comprising polypropylene.

A18. The product of example A17 wherein the polypropylene constitutes 20 to 40 weight percent of the second layer, and preferably no greater than 30 weight percent, more preferably no greater than 25 weight percent, and even more preferably no greater than 22 weight percent.

A19. The product of any of examples A15-A18 wherein the second layer constitutes 50 percent, preferably 55 percent or more, of a total basis weight of the film.

### B. Process and Equipment

B1. Equipment configured to simultaneously effect cross direction formation of seams and severing apart of individual seamed envelope packages comprising:

a first roller comprising a first seaming and severing element; and

a second roller comprising a second seaming and severing element,

wherein the first and second rollers have axes of rotation oriented along a cross direction;

wherein the first roller, the second roller, the first seaming and severing element and the second seaming and severing element are configured such that the first seaming and severing element will contact and oppose the second seaming and severing element along a line of contact oriented in the cross direction, in a transitory nip between the rollers; and

wherein at least one of the first roller, the second roller, the first seaming and severing element and the second seaming and severing element is associated with a pliant mechanism configured to enable one or both of the first seaming and severing element and the second seaming and severing element to displace radially toward the axis of rotation of its associated roller, in response to radial force exerted by the opposing seaming and severing element in the transitory nip.

B2. The equipment of example B1 wherein at least one of the first and second seaming and severing elements is a seaming and severing knife.

B3. The equipment of example B2 wherein the seaming and severing knife has an edge radius kr of 2 mm to 5 mm.

B4. The equipment of either of examples B2 or B3 wherein another of the first and second seaming and severing elements is an anvil.

B5. The equipment of example B4 wherein the anvil has a substantially flat acting surface opposing seaming and severing knife in the transitory nip.

B6. The equipment of any of the preceding examples wherein at least one of the first roller, the second roller, the first seaming and severing element and the second seaming and severing element is associated with a heating element configured to heat acting surface(s) of the first seaming and severing element and/or the second seaming and severing element.
B7. A process for forming individual packages containing a product, the packages being formed of polymeric film, comprising the steps of:

effecting phased spacing of individual quantities of product being conveyed along a machine direction, such that the individual quantities of product are regularly spaced apart along the machine direction;

conveying the spaced individual quantities of product along the machine direction, into a flow wrapping unit;

flow wrapping the spaced individual quantities in the polymeric film along the machine direction;

conveying the flow-wrapped, spaced individual quantities into a seaming and severing unit, wherein the seaming and severing unit comprises:
a first roller comprising a first seaming and severing element (318); and
a second roller comprising a second seaming and severing element,
wherein the first and second rollers have axes of rotation oriented along a cross direction;

wherein the first roller, the second roller, the first seaming and severing element and the second seaming and severing element are configured such that the first seaming and severing element will contact and oppose the second seaming and severing element along a line of contact oriented in the cross direction, in a transitory nip between the rollers; and

wherein at least one of the first roller, the second roller, the first seaming and severing element and the second seaming and severing element is associated with a pliant mechanism configured to enable one or both of the first seaming and severing element and the second seaming and severing element to displace radially toward the axis of rotation of its associated roller, in response to radial force exerted by the opposing seaming and severing element in the transitory nip;

conveying the flow-wrapped, spaced individual quantities into the transitory nip, and thereby:

compressing first and second portions of the polymeric film together in the transitory nip and plasticly expressing material thereof away from the transitory nip along the machine direction, thereby forming affixed regions wherein the first and second portions of the film are affixed to each other, and simultaneously, respective leading and trailing packages are effectively severed from one another.
B8. The process of example B7 wherein at least one of the first and second seaming and severing elements is a seaming and severing knife.
B9. The process of example B8 wherein the seaming and severing knife has an edge radius kr of 2 mm to 5 mm.
B10. The process of either of examples B8 or B9 wherein another of the first and second seaming and severing elements is an anvil.
B11. The process of example B10 wherein the anvil has a substantially flat acting surface opposing the seaming and severing knife in the transitory nip.
B12. The process of any examples B7-B11 wherein at least one of the first roller, the second roller, the first seaming and severing element and the second seaming and severing element is associated with a heating element configured to heat acting surface(s) of the first seaming and severing element and/or the second seaming and severing element.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. (canceled)

2. (canceled)

3. (canceled)

4. (canceled)

5. (canceled)

6. (canceled)

7. A process for forming individual packages containing a product, the packages being formed of polymeric film, comprising the steps of:
effecting phased spacing of individual quantities of product being conveyed along a machine direction, such that the individual quantities of product are regularly spaced apart along the machine direction;
conveying the spaced individual quantities of product along the machine direction, into a flow wrapping unit;
flow wrapping the spaced individual quantities in the polymeric film along the machine direction;
conveying the flow-wrapped, spaced individual quantities into a seaming and severing unit, wherein the seaming and severing unit comprises:
a first roller comprising a first seaming and severing element; and
a second roller comprising a second seaming and severing element,
wherein the first and second rollers have axes of rotation oriented along a cross direction;
wherein the first roller, the second roller, the first seaming and severing element and the second seaming and severing element are configured such that the first seaming and severing element will contact and oppose the second seaming and severing element along a line of contact oriented in the cross direction, in a transitory nip between the rollers; and
wherein at least one of the first roller, the second roller, the first seaming and severing element and the second seaming and severing element is associated with a pliant mechanism configured to enable one or both of the first seaming and severing element and the second seaming and severing element to displace radially toward the axis of rotation of its associated roller, in response to radial force exerted by the opposing seaming and severing element in the transitory nip;
conveying the flow-wrapped, spaced individual quantities into the transitory nip, and thereby:
compressing first and second portions of the polymeric film together in the transitory nip and plasticly expressing material thereof away from the transitory nip along the machine direction, thereby forming affixed regions wherein the first and second portions of the film are affixed to each other, and simultaneously, respective leading and trailing packages are effectively severed from one another.

8. The process of claim 7 wherein at least one of the first and second seaming and severing elements is a seaming and severing knife.

9. The process of claim 8 wherein the seaming and severing knife has an edge radius kr of 2 mm to 5 mm.

10. The process of claim 8 wherein another of the first and second seaming and severing elements is an anvil.

11. The process of claim 10 wherein the anvil has a substantially flat acting surface opposing seaming and severing knife in the transitory nip.

12. The process of claim 7 wherein at least one of the first roller, the second roller, the first seaming and severing element and the second seaming and severing element is associated with a heating element configured to heat acting surface(s) of the first seaming and severing element and/or the second seaming and severing element.
